(19) United States
(12) Patent Application Publication
(10) Pub. No.: US 2025/0171743 A1
(43) Pub. Date: May 29, 2025

(54) HUMAN TOOTH ORGANOIDS

(71) Applicants: Katholieke Universiteit Leuven, Leuven (BE); Universiteit Hasselt, Hasselt (BE)

(72) Inventors: Lara Hemeryck, Bali (ID); Hugo Vankelecom, Tervuren (BE); Annelies Bronckaers, Hasselt (BE); Ivo Lambrichts, Hasselt (BE)

(21) Appl. No.: 18/839,788

(22) PCT Filed: Feb. 22, 2023
(86) PCT No.: PCT/EP2023/054422

§ 371 Date: Aug. 20, 2024

(30) Foreign Application Priority Data
Feb. 22, 2022 (EP) 22158089.7

(51) Int. Cl. C12N 5/0775

(52) U.S. Cl. CPC C12N 5/0664; C12N 2500/30; C12N 2500/90; C12N 2501/01; C12N 2501/105; C12N 2501/115; C12N 2501/119; C12N 2501/415; C12N 2533/90

(57) ABSTRACT

The invention relates to method for developing and growing tooth organoids comprising the steps of, dissociating tooth tissue comprising dental epithelial stem cells, seeding the dissociated cells in a scaffold mimicking an extracellular matrix; and growing and passaging the cells in a medium suitable for organoid growth, wherein the medium does not comprise epidermal growth factor (EGF), thereby obtaining and expanding organoids.

### FIELD OF THE INVENTION

The invention relates to media for cultivating tooth organoids. The present invention further relates to tooth organoids and markers to characterise such organoids.

### INTRODUCTION

Teeth play essential roles in food mastication and speech. Moreover, tooth physiology is more and more highlighted to impact body health and disease. In contrast to the wealth of knowledge on tooth development, homeostatic maintenance and repair in rodents, tooth biology remains far from understood in humans. Although stem cells of the mesenchymal compartments such as dental pulp and periodontal ligament (PDL) have substantially been characterized, knowledge on human tooth epithelial stem cells regarding presence, phenotype and biological function is scarce [Yu & Klein (2020) Development 147, dev184754]. Some indications for their existence have been found in the Epithelial Cell Rests of Malassez (ERM), a network of epithelial cells that is present in the dental follicle (dental follicle) which encloses unerupted teeth and upon tooth eruption remains present in the periodontal ligament around the root [Davis (2018) J. Vet. Dent. 35, 290-298]. These nests of epithelial cells express some stem cell-associated markers, and may play a role in regeneration of enamel and periodontal ligament following injury and inflammation, although repair capacity appears limited in postnatal life [Davis (2018) J. Vet. Dent. 35, 290-298]. During tooth development, enamel is formed by epithelial cells called ameloblasts [Yu & Klein (2020) Development 147, dev184754]. It has been reported that epithelial cell rests of Malassez-derived cells, when co-cultured with dental pulp stem cells (DPSCs), can differentiate into ameloblast-like cells [Shinmura et al. (2008) J. Cell. Physiol. 217, 728-738]. However, 2D-cultured epithelial cell rests of Malassez show highly limited growth capacity and rapid loss of phenotype [Athanassiou-Papaefthymiou et al. (2015) J. Dent. Res. 94, 1591-1600; Kim et al. (2020) Int. J. Mol. Sci. 21, 1-16; Nam et al. (2014) Mol. Cells 37, 562-567; Nam et al. (2011) Mol. Cells 31, 355-36; Tsunematsu et al. (2016) Lab. Investig. 96, 1063-1075].

A powerful method to in vitro grow and expand tissue epithelial stem cells is provided by organoid technology. Although meanwhile derived from numerous organs, epithelial organoids have not been established yet from human tooth [Gao et al. (2021) J. Dental Res. 100, 454-463; Binder et al. (2020) Sci. Rep. 10, 4963]. A previous study reported that epithelial cell rests of Malassez, seeded in Matrigel, grew as ‘organoids’ [Athanassiou-Papaefthymiou et al. (2015) J. Dent. Res. 94, 1591-1600]. However, these structures were not deeply characterized, and did not adhere to the current hallmarks of tissue-derived organoids such as (clonal) derivation and expansion from epithelial tissue stem cells under WNT-promoting conditions, and robust and long-term expandability. Other studies described the construction of bioengineered 3D dental structures, however only from animal origin (mouse, rat, dog, pig) at embryonic or neonatal age and non-expandable [Nakao et al. (2007) Nat. Methods 4, 227-230; Duailibi et al. (2008) J. Dent. Res. 87, 745-750; Young et al. (2002) J. Dent. Res. 81, 695-700].

### SUMMARY OF THE INVENTION

The present invention discloses the establishment of long-term expandable organoid cultures starting from human tooth (i.e. from the dental follicle of third molars). The organoids show epithelial stemness characteristics mirroring epithelial cell rests of Malassez stem cells, and display ameloblast differentiation property reinforced by the presence of TGFb or dental mesenchymal cells, thereby recapitulating epithelial cell rests of Malassez/dental epithelial stem cell (DESC) features and known in vivo processes. These organoid models provide a valuable research tool to explore human tooth epithelial stem cell biology and epithelium-mesenchyme interplay, at present only poorly understood, thereby paving the way to unravelling their roles in tooth homeostasis and potential repair. Moreover, the tractable biological tooth stem cell structures represent an appealing step toward dental regenerative replacement prospects.

Insight into human tooth epithelial stem cells and their biology is sparse. Tissue-derived organoid models typically replicate the tissue's epithelial stem cell compartment. The present invention discloses an epithelial organoid model starting from human tooth. Dental follicle (dental follicle) tissue, isolated from unerupted wisdom teeth, efficiently generated epithelial organoids that were long-term expandable. The organoids displayed a tooth epithelial stemness phenotype similar to the dental follicle's Epithelial Cell Rests of Malassez (ERM), a compartment containing dental epithelial stem cells. Single-cell transcriptomics reinforced this organoid-epithelial cell rests of Malassez congruence, and uncovered novel, mouse-mirroring stem cell features. Exposure of the organoids to epidermal growth factor induced transient proliferation and eventual epithelial-mesenchymal transition, highly mimicking events taking place in the epithelial cell rests of Malassez in vivo. Moreover, the epithelial cell rests of Malassez stemness organoids were able to unfold an ameloblast differentiation process, further enhanced by transforming growth factor-beta (TGFβ) and abrogated by TGFβ receptor inhibition, thereby reproducing TGFβ's known key position in amelogenesis. By creating a mesenchymal-epithelial composite organoid (assembloid) model, it is herein demonstrated that the presence of dental mesenchymal cells (i.e. pulp stem cells) triggered ameloblast differentiation in the epithelial stem cells, thus replicating the known importance of mesenchyme-epithelium interaction in tooth development and amelogenesis. Also here, differentiation was abrogated by TGFβ receptor inhibition. Novel organoid models are described, empowering the exploration of human tooth epithelial stem cell biology and function as well as their interplay with dental mesenchyme, all at present only poorly defined in humans. Moreover, the new models may pave the way to future tooth-regenerative perspectives.

The invention is further summarized in the following statements:
1. A method for developing and growing tooth organoids comprising the steps of:
   dissociating tooth tissue comprising dental epithelial stem cells;
   seeding the dissociated cells in a scaffold mimicking an extracellular matrix; and
   growing and passaging the cells in a medium suitable for organoid growth, wherein the medium does not comprise epidermal growth factor (EGF), thereby obtaining and expanding organoids. Typically the media used in the methods of the present invention are serum free.
2. The method according to statement 1, wherein the medium comprises:
   a WNT agonist,
   a key nicotinamide adenine dinucleotide (NAD+) intermediate,
   an activin receptor-like kinase (ALK) inhibitor,
   a p38 MAP kinase inhibitor,
   a bone morphogenetic protein (BMP) inhibitor,
   a fibroblast growth factor (FGF),
   an insulin-like growth factor,
   a free-radical scavenger,
   a hedgehog signalling agonist,
   an adenylate cyclase-CAMP agonist,
   B27,
   L-glutamine, and
   N2.
3. The method according to statement 2, wherein:
   the WNT agonist is R-spondin 1 (RSPO1) or WNT3A,
   the key nicotinamide adenine dinucleotide (NAD+) intermediate is nicotinamide,
   the activin receptor-like kinase (ALK) inhibitor is A83-01,
   the p38 MAP kinase inhibitor is SB202190,
   the bone morphogenetic protein (BMP) inhibitor is Noggin,
   the fibroblast growth factor (FGF) is FGF8, FGF10, and FGF2,
   the insulin-like growth factor is IGF-1,
   the free-radical scavenger is N-acetyl-cysteine (NAC),
   the hedgehog signalling agonist is Sonic hedgehog (SHH),
   the adenylate cyclase-CAMP agonist is cholera toxin.
4. The method according to any one of statements 1 to 3, wherein the medium comprises:
   between 150 and 250 ng/ml, or between 175 and 225 ng/ml, or 200 ng/ml R-spondin 1 (RSPO1) and between 150 and 250 ng/ml, or between 175 and 225 ng/ml, or 200 ng/ml WNT3A,
   between 7.5 and 12.5 mM, between 9 and 11 mM or 10 mM nicotinamide,
   between 0.4 and 0.6 μM, between 0.45 and 0.55 UM or 0.5 μM A83-01,
   between 7.5 and 12.5 μM, between 9 and 10 UM or 10 UM SB202190,
   between 75 and 125 ng/ml, between 90 and 110 ng/ml or 100 ng/ml Noggin,
   between 150 and 250 ng/ml, or between 175 and 225 ng/ml, or 200 ng/ml FGF8,
   between 75 and 125 ng/ml, between 90 and 110 ng/ml or 100 ng/ml FGF10,
   between 15 and 25 ng/ml, or between 17.5 and 22.5 ng/ml, or 20 ng/ml FGF2,
   between 75 and 125 ng/ml, between 90 and 110 ng/ml or 100 ng/ml IGF-1,
   between 0.937 and 1.563 mM, between 1.125 and 1.375 mM or 1.25 mM N-acetyl-cysteine (NAC),
   between 75 and 125 ng/ml, between 90 and 110 ng/ml or 100 ng/ml SHH,
   between 75 and 125 ng/ml, between 90 and 110 ng/ml, or 100 ng/ml cholera toxin,
   between 1.75 and 2.25%, between 1.9% and 2.1%, or 2% B27,
   between 1.5 and 2.5 mM, between 1.75 and 2.25 mM or 2 mM L-glutamine, and
   between 0.75 and 1.25%, between 0.9 and 1.1%, or 1% N2.
5. The method according to any one of statements 1 to 4, wherein the stem cells are isolated from dental follicle or from dental periodontal ligament.
6. The method according to any one of statements 1 to 4, wherein the stem cells are tooth epithelial stem cells isolated from dental follicle, periodontal ligament of third molars (wisdom teeth) or molars.
7. The method according to any one of statements 1 to 6, wherein the organoids are passaged for at least one to more than 10 passages, by dissociating the organoids into single cells and cultivating the single cells in said medium without EGF.
8. The method according to any one of statements 1 to 7, further culturing the organoids in a medium suitable for organoids, comprising EGF, thereby inducing mesenchymal properties.
9. The method according to any one of statements 1 to 7, wherein the organoids are further cultured in a medium comprising transforming growth factor beta (TGFβ), thereby enhancing amelogenesis and periodontal ligament differentiation.
10. The method according to statement 9, wherein the medium comprises a keratinocyte serum-free medium, calcium and bovine pituitary extract
11. A tooth organoid comprising epithelial cells from tooth tissue and expressing amelogenin (AMELX).
12. A tooth organoid according to statement 11, obtainable by the method according to any one of statements 1 to 10.
13. The organoid according to statement 11 or 12, which does not express ODAM.
14. The organoid according to any one of statements 11 to 13, which does not express one or more of CD90, fibroblast activation protein alpha (FAP) and Collagen Type I alpha I (COL1A1).
15. The organoid according to any one of statements 11 to 14, further expressing cytokeratin 14 (CK14), 5 (CK5).
16. The organoid according to any one of statements 11 to 15, further expressing one or more of CD44, TP63, SOX2, ITGA6, BMP4 and KRT15.
17. A differentiated tooth organoid comprising epithelial cells from tooth tissue and producing electron-dense calcium-phosphate accumulations, expressing ODAM and AMELX.
18. The differentiated tooth organoid according to statement 17, further staining positive for Alizarin Red Staining.
19. The differentiated tooth organoid according to statement 17 or 18, further expressing AMTN, KLK4 and CK19.
20. The differentiated tooth organoid according to any one of statements 17 to 20, further expressing one or more of LAMC2, LAMA3, LAMB3, FDCSP, STIM1, CALB2 and TGFβI.
21. A hybrid organoid comprising epithelial cells from tooth tissue and mesenchymal cells from dental tissue (pulp) producing electron-dense calcium-phosphate accumulations.
22. The organoid according to statement 22, expressing ODAM and AMTN.
23. A medium for the development, growth and culture of epithelial organoids from human tooth tissue, wherein the medium does not comprise epidermal growth factor (EGF), and wherein the medium comprises:
   a WNT agonist,
   a key nicotinamide adenine dinucleotide (NAD+) intermediate,
   an activin receptor-like kinase (ALK) inhibitor,
   a p38 MAP kinase inhibitor,
   a bone morphogenetic protein (BMP) inhibitor,
   a fibroblast growth factor (FGF),
   an insulin-like growth factor,
   a free-radical scavenger,
   a hedgehog signalling agonist,
   an adenylate cyclase-CAMP agonist,
   B27,
   L-glutamine and
   N2.
24. The medium according to statement 23, wherein
   the WNT agonist is R-spondin 1 (RSPO1) or WNT3A,
   the key nicotinamide adenine dinucleotide (NAD+) intermediate is nicotinamide,
   the activin receptor-like kinase (ALK) inhibitor is A83-01,
   the p38 MAP kinase inhibitor is SB202190,
   the bone morphogenetic protein (BMP) inhibitor is Noggin,
   the fibroblast growth factor (FGF) is FGF8, FGF10 and FGF2,
   the insulin-like growth factor is IGF-1,
   the A free-radical scavenger is N-acetyl-cysteine (NAC),
   the hedgehog signalling agonist is Sonic hedgehog (SHH),
   the adenylate cyclase-CAMP agonist is cholera toxin.
25. The medium according to statement 24 comprising:
   between 150 and 250 ng/ml, or between 175 and 225 ng/ml, or 200 ng/ml R-spondin 1 (RSPO1) or comprising between 150 and 250 ng/ml, or between 175 and 225 ng/ml, or 200 ng/ml WNT3A,
   between 7.5 and 12.5 mM, between 9 and 11 mM or 10 mM nicotinamide,
   between 0.4 and 0.6 μM, between 0.45 and 0.55 UM or 0.5 μM A83-01,
   between 7.5 and 12.5 μM, between 9 and 10 UM or 10 μM SB202190,
   between 75 and 125 ng/ml, between 90 and 110 ng/ml or 100 ng/ml Noggin,
   between 150 and 250 ng/ml, or between 175 and 225 ng/ml, or 200 ng/ml FGF8,
   between 75 and 125 ng/ml, between 90 and 110 ng/ml or 100 ng/ml FGF10,
   between 15 and 25 ng/ml, or between 17.5 and 22.5 ng/ml, or 20 ng/ml FGF2,
   between 75 and 125 ng/ml, between 90 and 110 ng/ml or 100 ng/ml IGF-1,
   between 1 and 1.5 mM, between 1.15 and 1.35 mM or 1.25 mM N-acetyl-cysteine (NAC),
   between 75 and 125 ng/ml, between 90 and 110 ng/ml or 100 ng/ml SHH,
   between 75 and 125 ng/ml, between 90 and 110 ng/ml or 100 ng/ml cholera toxin,
   between 1.75 and 2.25%, between 1.9% and 2.1%, or 2% B27,
   between 1.5 and 2.5 mM, between 1.75 and 2.25 mM or 2 mM L-glutamine, and
   between 0.75 and 1.25%, between 0.9 and 1.1% or 1% N2.
26. Use of a medium according to any one of statements 23 to 25, for the development, growth and culture of epithelial organoids from human tooth tissue

### FIGURES

FIG. 1 Establishment of organoids from human dental follicle

a Schematic of organoid culture set-up. Progressing development of organoid structures after seeding dissociated dental follicle (DF) in tooth organoid medium (TOM) (passage 0, P0), and robust passageability (brightfield pictures of indicated P). b Histological (H&E) and ultrastructural (TEM) analyses of tooth organoids grown in TOM for 14 days. Box and arrow indicate cuboidal epithelium (CE) and squamous epithelium (SE), respectively. c-e Brightfield phase-contrast images and immunofluorescence staining pictures for markers as indicated, of primary dental follicle tissue and full-grown (day-14) organoids. Arrows indicate double-positive cells of indicated markers. Boxed areas are enlarged. DAPI was used to label nuclei. Scale bars: 50 μm, unless indicated otherwise.

FIG. 2 Single-cell transcriptomic profiling of primary dental follicle and corresponding organoids

a Experimental overview of the scRNA-seq analysis. UMAP plot of the annotated cell clusters in the integrated dental follicle-organoid dataset. DF, dental follicle; ERM, Epithelial Cell Rests of Malassez; NK, natural killer cells. b Heatmap displaying the scaled expression of the top 10 differentially expressed genes (DEGs) per cluster. Genes specifically described in the text are highlighted in bold. c Dot plot displaying the percentage of cells (dot size) expressing indicated marker genes with average expression levels (colour intensity) (see scales) in the epithelial cell rests of Malassez and organoid clusters. d Indicated regulons projected on UMAP plot. The ERM cluster is magnified at the bottom. e Immunofluorescence staining for markers as indicated in primary dental follicle tissue and organoids at specified time points. DAPI (blue) was used to label nuclei. f Significant (FDR≤0.05) DEG-based GO terms enriched in epithelial cell rests of Malassez versus P1 organoids (top) or in P1 and P4 organoids together versus epithelial cell rests of Malassez (bottom). g Violin plots showing gene expression level of indicated stemness markers in P1 and P4 organoids. Immunofluorescence staining of P1 and P4 organoids for the indicated markers. DAPI (blue) was used to label nuclei. Scale bars: 50 μm.

FIG. 3 Effect of EGF on tooth organoid culture

a Timeline of experimental set-up (d, day). Immunofluorescence analysis and quantification of KI67⁺ cells (mean±SEM; n=3 biological replicates) in organoids cultured as indicated. DAPI (blue) was used to label nuclei. b Immunofluorescence staining for the indicated markers of full-grown organoids (day 14; P0) cultured in medium as denoted. Arrows indicate double VIM⁺CK5⁺ cells. DAPI (blue) was used to label nuclei. c Timeline of experimental set-up. Left part: brightfield pictures of organoid cultures (day 14) as indicated. Boxed area is enlarged. Encircled areas show cell growth at the bottom of the culture plate. Immunofluorescence staining of full-grown organoids (day 14; P5) cultured as indicated for the indicated markers. Right part: brightfield pictures and immunofluorescence (VIM) staining of cells grown at the bottom of the plate (day 14; P5). Boxed area is enlarged. DAPI (blue) was used to label nuclei. Asterisk mark for orientation. Scale bars: 50 μm, unless indicated otherwise.

FIG. 4 Ameloblast differentiation-mimicking process in tooth organoids

a Timeline of experimental set-up (d, day). Immunofluorescence examination of ODAM in organoids from culture conditions and time points as indicated. DAPI (blue) was used to label nuclei. Corrected total organoid fluorescence (CTOF) quantification of ODAM in organoids at indicated time points (mean±SEM; n=3 biological replicates). b Gene expression pattern of ameloblast secretory- and maturation-stage markers in MIM-switched organoids at time points as indicated. Data are expressed as fold change relative to the organoids at switching to MIM (d0). Expression is normalized to expression of GAPDH. Data are mean of n=3 biological replicates. Right: Gene expression levels (relative to GAPDH) of AMTN in MIM-switched organoids at time points as indicated (mean±SEM; n=3 biological replicates). Below: Immunofluorescence staining for the indicated markers, and quantification of SOX2⁺ and P63⁺ cells in organoids cultured in MIM (mean±SEM; n=3 biological replicates). DAPI (blue) was used to label nuclei. c Immunofluorescence staining for the indicated markers in organoids cultured as specified. DAPI (blue) was used to label nuclei. d Alizarin Red S (ARS) staining of organoids cultured as specified. Arrows indicate ARS⁺ areas. Images below show negative control (i.e. hematoxylin only). Right: Ultrastructural (TEM) analysis of MIM-switched organoids. Boxed area is enlarged. Arrowheads indicate calcium phosphate crystals. Scale bars: 50 μm, unless indicated otherwise.

FIG. 5 Single-cell transcriptomic profiling of tooth organoids driven into amelogenesis-resembling differentiation

a Experimental overview of the scRNA-seq analysis. UMAP plot of the integrated dental follicle and organoid samples as indicated. ‘Primary’ means all dental follicle clusters. b Projection of indicated genes on the integrated UMAP plot. c Heatmap displaying the scaled expression of the top 10 DEGs per cluster in P4 versus P4-switch organoids. d Significant (FDR≤0.05) DEG-based GO terms enriched in P4-switch versus P4 organoids. e Indicated regulons (STAT2, MAF, FOXC2) projected on the integrated UMAP plot. Dot plot of predicted STAT2 or MAF regulon target genes in P4 and P4-switch organoids. Projection of TGFβI gene expression on the UMAP plot.

FIG. 6 Effect of TGFβ on differentiation in tooth organoids

a Timeline of experimental set-up (d, day). Immunofluorescence examination for the indicated markers in organoids cultured as denoted. Boxed areas are enlarged. DAPI (blue) was used to label the nuclei. CTOF quantification of indicated markers in organoids cultured as specified (mean±SEM; n=3 biological replicates). b-c Gene expression levels (relative to GAPDH) of indicated markers in organoids cultured as denoted (mean±SEM; n>3 biological replicates). d Timeline of experimental set-up. Histological (H&E) analysis and immunofluorescence examination for the indicated markers in assembloids cultured as indicated. DAPI (blue) was used to label the nuclei. Dotted area demarcates the (VIM⁺) mesenchymal cells. Scale bars: 50 μm.

FIG. 7 Establishment of organoids from human dental follicle

a Brightfield images of the development of organoid structures (P0; day 14) after seeding dissociated dental follicle (DF) in the medium as indicated (see text). b Organoids growing out from dental follicle-derived cell clusters (top) or from single cells (bottom) in tooth organoid medium (TOM; passage 0, P0; d, day). c Histological (H&E) analysis of dental follicle. Boxed area is enlarged. Arrows indicate Epithelial Cell Rests of Malassez (ERM). d Brightfield and epifluorescence pictures of eGFP+ and eGFP− cells generated from dissociated organoids and cultured as mixture in TOM (P6; day 0 and day 14). Arrow indicates a fluorescent (eGFP+) organoid and arrowheads point to non-fluorescent (eGFP−) organoids. e Brightfield image of organoid culture after seeding of the dissociated dental follicle tissue in TOM (P0; day 14), showing attachment of spindle-formed mesenchymal cells at the bottom of the culture plate (arrows), which are lost at passaging, being not present anymore in the first passage (P1; day 14) Consecutive magnifications are indicated by black and blue boxes, respectively. f Progressing organoids' development in TOM during a single-passage (P4) 14-day culture period showing brightfield pictures, organoid diameters (mean±SEM; n=3 biological replicates), and proportions of proliferative and apoptotic events as quantified through KI67 and cleaved caspase-3 (CC3) immunostaining, respectively (dots indicate biological replicates; n=2). g Proportion of apoptotic (CC3+) cells and diameters of day-14 organoids over different passages (mean±SEM; n=3 biological replicates). h Organoids derived from dental follicle of erupted wisdom teeth from patients as indicated. i Immunofluorescence staining for markers as indicated in primary dental follicle tissue and organoids (P0, day 14). DAPI was used to label nuclei. j Gene expression levels (relative to GAPDH) of indicated markers in dental follicle and organoids (P1) (mean±SEM; n=3 biological replicates). Scale bars: 50 μm, unless indicated otherwise.

FIG. 8 Single-cell transcriptomic analysis of primary dental follicle and corresponding organoids

a Dot plot displaying the percentage of cells (dot size) expressing indicated marker genes with average expression levels (colour intensity) (see scales) of the annotated cell clusters. UMAP representation of the distinct cell clusters, and UMAP plot of the different patients (Pat). b Violin plots showing the distribution of the number of genes detected per cell (nGene), the total unique molecular identifier counts per cell (nUMI) and the percentage of mitochondrial content (percent.mito) per sequenced sample as indicated. Dashed lines show cut-off values (see Methods). c Significant (FDR≤0.05) DEG-based GO term enriched in the lower-quality cell cluster based on the top 10 DEGs. Ultrastructural (TEM) analysis of full-grown organoids (P5; day 15). Boxed area is enlarged. Arrowhead indicates an apoptotic nucleus. d-e Projection of indicated genes on UMAP plot. epithelial cell rests of Malassez cluster is enlarged at the bottom. f Projection of ITGA6 expression on UMAP plot. epithelial cell rests of Malassez cluster is enlarged at the bottom. Brightfield pictures of organoid cultures from FACS-isolated ITGα6+ or ITGα6− cells in TOM (P0; day 17). Boxed area is enlarged. Arrows indicate attached spindle-formed mesenchymal cells in the ITGα6− cell culture at the bottom of the plate. g Projection of indicated genes on UMAP plot. epithelial cell rests of Malassez cluster is enlarged at the bottom. Immunofluorescence staining of dental follicle and (day-14) organoids for indicated markers. DAPI (blue) was used to label nuclei. h Violin plots displaying activity of indicated regulons in the epithelial cell rests of Malassez and organoid clusters. Scale bars: 50 μm, unless indicated otherwise.

FIG. 9 Effect of EGF on tooth organoid cultures

a Projection of indicated genes on UMAP plot. b Timeline of experimental set-up (d, day) and brightfield pictures of organoid cultures (day 14) as indicated. Right: diameter of organoids in specified cultures (violin plot; n=3 biological replicates). c Immunofluorescence staining for indicated markers in organoids cultured in TOM+EGF (P0). Boxed area is enlarged. DAPI (blue) was used to label nuclei. Arrows indicate double P63+VIM+ cells. d Expression levels (relative to GAPDH) of indicated genes in organoids (day 14) cultured as denoted (mean±SEM; n=4 biological replicates). Scale bars: 50 μm.

FIG. 10 Ameloblast differentiation-mimicking process in tooth organoids

a Immunofluorescence staining for AMELX in organoids cultured as denoted. DAPI (blue) was used to label nuclei. b Gene expression levels (relative to GAPDH) of indicated markers in organoids cultured in TOM at indicated time points (mean #SEM; n=3 biological replicates). c Timeline of in vivo experimental set-up (d, day). ARS and Masson's trichrome (TCM) staining of depositions in recovered hydroxyapatite scaffolds which had been seeded with organoids before subcutaneous implantation, or only with Matrigel (empty). Boxed areas are enlarged. Negative control of ARS involves hematoxylin staining only. Arrow indicates a still discernible organoid. d Timeline of experimental set-up (d, day). Immunofluorescence staining for indicated markers in full-grown organoids as specified. DAPI (blue) was used to label nuclei. Quantification of SOX2+ cells in organoids cultured as indicated (mean±SEM; n=3 biological replicates). Gene expression levels (relative to GAPDH) of indicated markers in organoids cultured as specified (mean±SEM; n=3 biological replicates). Scale bars: 50 μm, unless indicated otherwise.

FIG. 11 Analysis of scRNA-seq data of tooth organoids driven into amelogenesis-resembling differentiation

a Projection of indicated genes on the integrated UMAP plot (see FIG. 5a). b Expression levels (relative to GAPDH) of indicated genes in organoids cultured as specified (mean±SEM; n=4 biological replicates). c Significant (FDR≤0.05) DEG-based GO terms enriched in P4 versus P4-switch organoids. d Indicated regulons projected on the integrated UMAP plot. Dot plot of predicted HMGA2 regulon target genes in P4 and P4-switch organoids. e Pseudotime projected onto the integrated UMAP plot. Encircled area indicates a subcluster of potential transitional stage. f Indicated regulons projected on the integrated UMAP plot. g STRING protein-protein interaction network generated from the top 40 DEGs in P4-switch versus P4 organoids, predicting associations between proteins (nodes). The cluster analysis was subdivided in three colours by kmeans. Thickness of connecting line indicates confidence of interaction. Genes specifically described in the text are highlighted in bold. h Significant (FDR≤0.05) DEG-based GO terms enriched in top 40 DEGs of P4-switch versus P4 organoids by Biological Process and KEGG Pathway analysis.

FIG. 12 Effect of TGFβ on differentiation in tooth organoids and assembloids

a Gene expression analysis of indicated TGFβ pathway components in organoids cultured as denoted. Expression is normalized to expression of GAPDH. Data are mean±SEM of n=3 biological replicates. b Brightfield and fluorescent (eGFP) images of assembloids comprising g dental epithelial (organoid-derived) cells and mesenchymal cells (DPSCs; marked by eGFP), cultured in TOM+αMEM. c Time-line of experimental set-up (d, day). Immunofluorescence staining for indicated markers of organoids cultured as denoted. DAPI (blue) was used to label the nuclei. Scale bar: 200 μm, unless indicated otherwise. d-e Gene expression levels (relative to GAPDH) of indicated TGFβ pathway components in assembloids cultured in TOM+□MEM (mean±SEM; n=3 biological replicates).

Organoids are 3D cell constructs that self-develop by proliferative expansion from tissue's epithelial stem cells when the dissociated primary tissue sample (containing the stem cells as single cells or contained within cell clusters) is seeded into an extracellular matrix (ECM)-mimicking scaffold (typically, Matrigel) and cultured in a defined cocktail of growth factors replicating stem cell niche signalling (if known) and/or tissue embryogenesis. Among others, activation of wingless-type MMTV integration site (WNT) and epidermal growth factor (EGF) signalling are typically needed [Boretto et al. (2017) Dev. 144, 1775-1786; Cox et al. (2019) J. Endocrinol. 240, 287-3089; Sato et al. (2009) Nature 459, 262-265]. Resultant organoids duplicate the epithelial stem cell compartment of the tissue of origin in molecular phenotype and functional characteristics, and can generate differentiated tissue cell types under specified culture condition. As an important asset, organoid cultures can be serially expanded (passaged) without loss of characteristics, thereby providing a robust and faithful source of the primary tissue's epithelial stem cells and overcoming their generally limited availability and culture-ability. Typically, epithelial organoid models are established without the need for prior isolation of the epithelial (stem) cells from the dissociated whole-tissue sample since the accompanying mesenchymal cells do not thrive in the specific culture conditions used and are swiftly lost at culture and passaging.

The present invention reports the development of a long-term expandable epithelial organoid model derived from human dental tissue. The dental follicle-derived organoids show a stemness expression profile congruent with the epithelial cell rests of Malassez, previously advanced to encompass dental epithelial stem cells [Davis (2018) J. Vet. Dent. 35, 290-298]. In addition, single-cell transcriptomics uncovered novel molecular features (such as the stemness-associated hybrid E/M nature, new markers and gene-regulatory networks) for the as yet ill-defined and poorly comprehended human dental epithelial stem cells and epithelial cell rests of Malassez, often mirroring findings in mouse. Noticeably, organoid culturing appeared to proliferatively (re-) activate the stem cells of epithelial cell rests of Malassez, previously reported to be highly quiescent in vivo [Shinmura et al. (2008) J. Cell. Physiol. 217, 728-738]. Moreover, described (stem cell-related) functional properties of the epithelial cell rests of Malassez were markedly recapitulated by the tooth organoids. First, exposure to EGF induced transient proliferation and eventual EMT and migration, thereby mimicking events taking place in the epithelial cell rests of Malassez in vivo (for instance, upon tooth insult) [Davis (2018) J. Vet. Dent. 35, 290-298]. Second, the tooth organoids displayed the capacity to unfold an ameloblast differentiation process, as occurring in vivo during tooth formation [Yu & Klein (2020) Development 147, dev184754] and reported for epithelial cell rests of Malassez [Hamamoto et al. (1996) Oral Surg. Oral Med. Oral Pathol. Oral Radiol. Endod., 81, 703-709], thus recapitulating the epithelial cell rests of Malassez differentiation capacity. The organoids displayed molecular changes constituting pathways that underlie ameloblast differentiation during amelogenesis [Liu et al., (2015) BMC Genomics 16, 592; Nurbaeva et al. (2017) J. Physiol. 595, 3015-3039]. In addition, the organoids recovered the key position of TGFβ in ameloblast differentiation/amelogenesis [Benedete (2008) Pediatr. Dev. Pathol. 11, 206-212], as well as in periodontal ligament development [Davis (2018) J. Vet. Dent. 35, 290-Moreover, the present scRNA-seq interrogation advanced molecular transitions not revealed before in human amelogenesis. Also, STRING analysis projected protein-protein interactions that may further deepen knowledge on amelogenesis in human tooth, at present not understood. Together, the present model has the potential to in detail decipher ameloblast development and their production of enamel, the quintessential component of teeth, which would represent a leap forward in the dental field (especially for future dental tissue replacement therapies). Third, the organoid transcriptome reflected functional processes before (provisionally) assigned to the epithelial cell rests of Malassez, including regulation of bone mineralization, osteoblast differentiation and tooth eruption. Hence, the present model provides a tool to help decipher the multiple biological functions assigned to the epithelial cell rests of Malassez. The organoids show strong expandability, thereby overcoming current hurdles of primary epithelial cell rests of Malassez/dental epithelial stem cell culturing, such as limited cell number, life span and phenotypical loss. The expansion ability will be highly instrumental for allowing in-depth analysis of this yet enigmatic cell population. Finally, the induction of ameloblast differentiation by the presence of mesenchymal cells, thereby recapitulating the acknowledged importance of epithelium-mesenchyme interaction in tooth development including amelogenesis, again further corroborated the biomimetic value of the present model(s). Altogether, the several characterizations provide strong evidence that the present human tooth (dental follicle)-derived organoid models, present a valuable tool to study human tooth epithelial stem-cell biology and development, at present far from understood. Organoid technology is also highly applicable to human disease modelling in vitro. It has been suggested that epithelial cell rests of Malassez cells are associated with the pathogenesis of odontogenic cysts and tumours. Developing organoids from these lesions may help to gain better insight in their pathogenesis. More in general, the present tooth organoid approach can harnessed to model and study tooth diseases ranging from impact of bacteria to genetic mutations (like mutations in P63 and PITX2 associated with tooth anomalies and amelogenesis imperfecta), eventually leading to novel therapeutic targets and treatments.

Organoids have been shown amenable to regenerative replacement therapy. Damaged, lost or missing teeth, causing major health problems, may be regenerated or replaced by transplanting biological tooth constructs. Such approach may be superior (both material- and function-wise) to the traditional, still suboptimal synthetic implants, among others suffering from lack of physiological functionality, inferior bone integration and absence of innervation. Embryonically derived, bioengineered mouse tooth germs have been shown capable of forming a functional tooth unit after transplantation in an emptied dental cavity of the mouse. The present organoid and assembloid models may provide essential puzzle pieces toward developing human tooth germs. Although transplantation of natural teeth has been performed in some patients, especially children and young adolescents, the availability of such teeth remains limited. Of important note, the murine Matrigel should then be replaced by a clinically compatible ECM mimic. Currently, attempts are being made to substitute Matrigel for defined synthetic hydrogels, although achievements are still limited. In conclusion, a long-term expandable stemness organoid model from human tooth is developed, replicating molecular and functional features of the originating epithelial stem cell compartment. The new in vitro model will be highly valuable to explore human tooth epithelial stem cell phenotype and biology such as ameloblast differentiation. Moreover, the present invention indicates that the postnatal human tooth still contains epithelial stem cells, and the organoids will be beneficial to address the question on their role(s), and on the reasons why they do not, or not prominently, regenerate tooth tissue in postnatal life. This search also implicates the question whether these stem cells can in vivo be re-activated for repair. This understanding may eventually instigate tooth-regenerative approaches by re-activating endogenous repair capacity and processes.

The present invention discloses, as illustrated by Example 1, that epithelial organoids can be established from human tooth-derived dental follicle, displaying an epithelial cell rests of Malassez-mirroring, stemness expression phenotype and possessing robust long-term expandability.

The present invention discloses, as illustrated by Example 2, the present detailed scRNA-seq interrogation demonstrates and reinforces the organoid-epithelial cell rests of Malassez stemness relationship and uncovered new molecular fingerprints of human epithelial cell rests of Malassez, at present only poorly defined.

The present invention discloses, as illustrated by Example 3, that adding EGF to the organoids recapitulates functional in vivo behaviour of the epithelial cell rests of Malassez, thus advancing the present tooth organoid model as an interesting tool to study epithelial cell rests of Malassez phenotype and conduct, to date not well understood.

The present invention discloses, as illustrated by Example 4, that the herein disclosed tooth organoid model is capable of unfolding an ameloblast differentiation process involving known consecutive steps, thereby recapitulating dental epithelial stem cell/epithelial cell rests of Malassez functionality, and thus provides a valuable research tool to study amelogenesis of human tooth, at present poorly defined.

The present invention discloses, as illustrated by Example 5, single-cell transcriptomics of the tooth organoids driven into amelogenesis differentiation demonstrates and underscores the relevance of the present organoid model by confirming known data as well as presenting new insights in the amelogenesis process in humans which is at present far from clarified.

The present invention discloses, as illustrated by Example 6, that TGFβ coerces the tooth organoids into more pronounced ameloblast differentiation as well as into the direction of periodontal ligament development. These findings conform to the known activity of TGFβ in these tooth developmental processes, and thus again corroborate the strength and validity of the present organoid model. Moreover, they provide supportive evidence that the dental follicle-derived organoids replicate the multipotency of dental (HERS/epithelial cell rests of Malassez) epithelial stem cells as proposed to unfold in vivo during tooth development and possibly repair.

The present invention discloses, as illustrated by Example 7, that ameloblast differentiation of epithelial (organoid) stem cells is triggered by the presence of tooth mesenchymal cells involving TGFβ signalling, thereby corroborating in vivo findings of interactive mesenchyme-epithelium importance, and further validating this model as valuable research tool for exploring human tooth (stem cell) biology.

### EXAMPLE 1. ORGANOIDS CAN BE ESTABLISHED FROM HUMAN DENTAL FOLLICLE

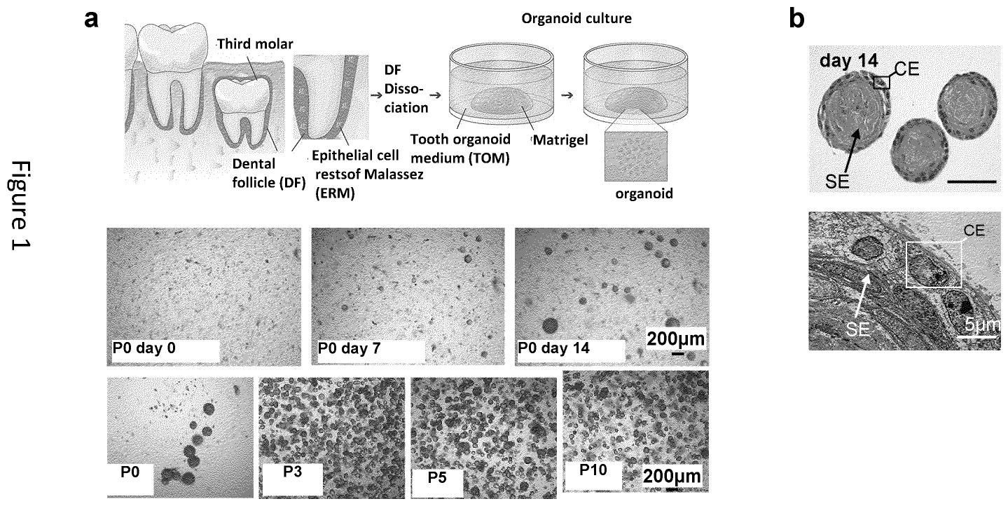

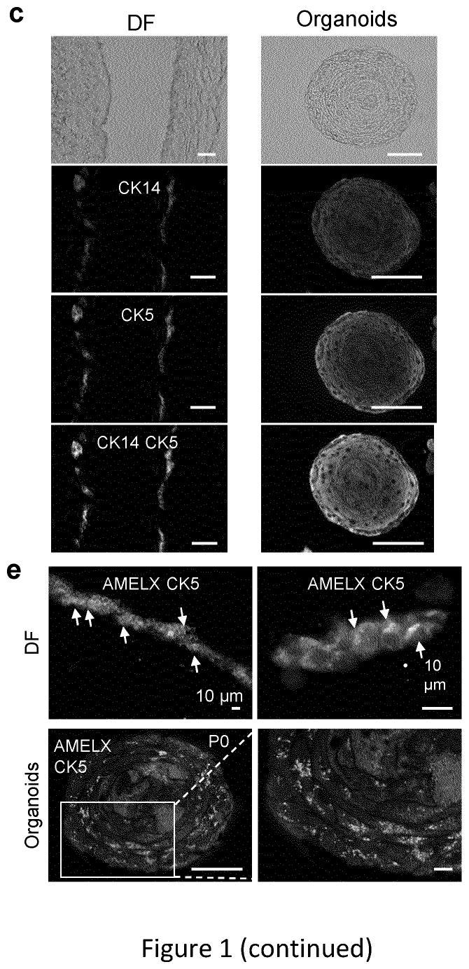

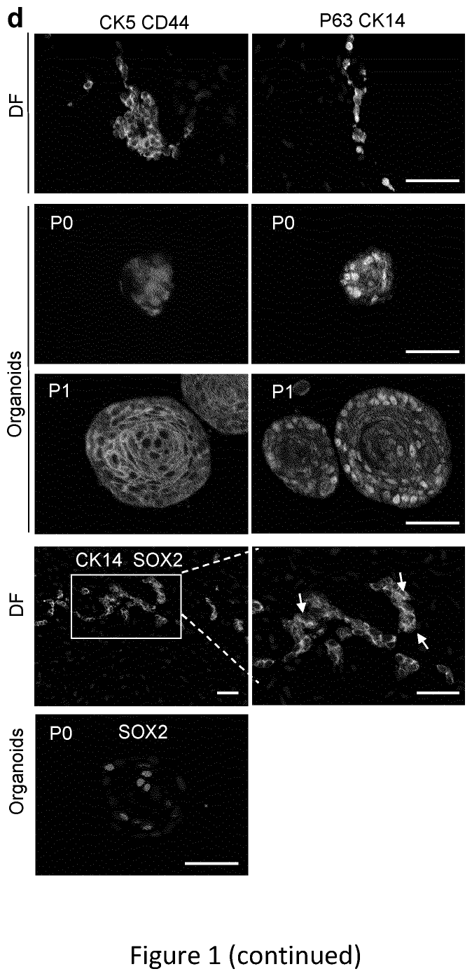

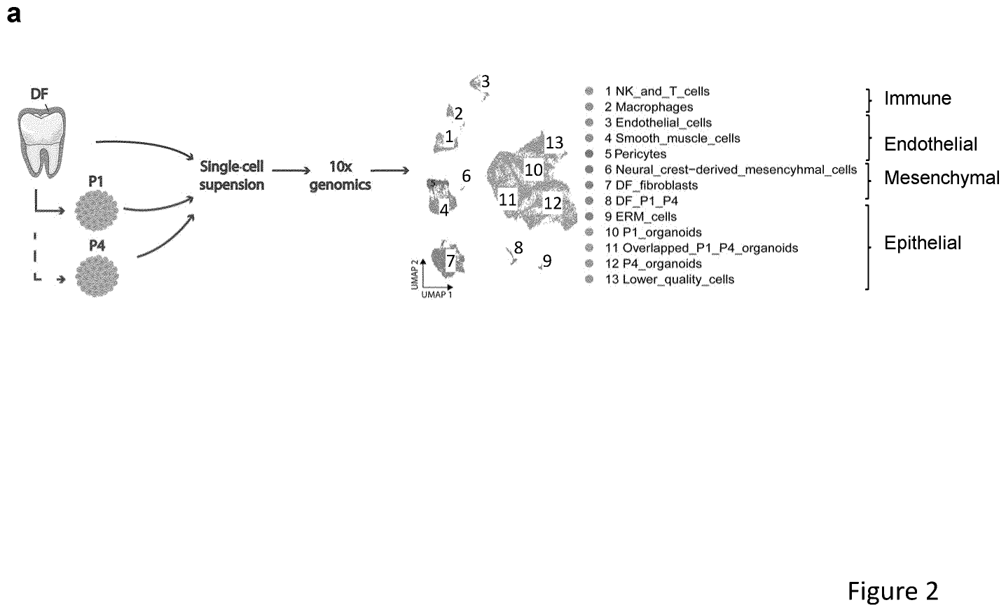

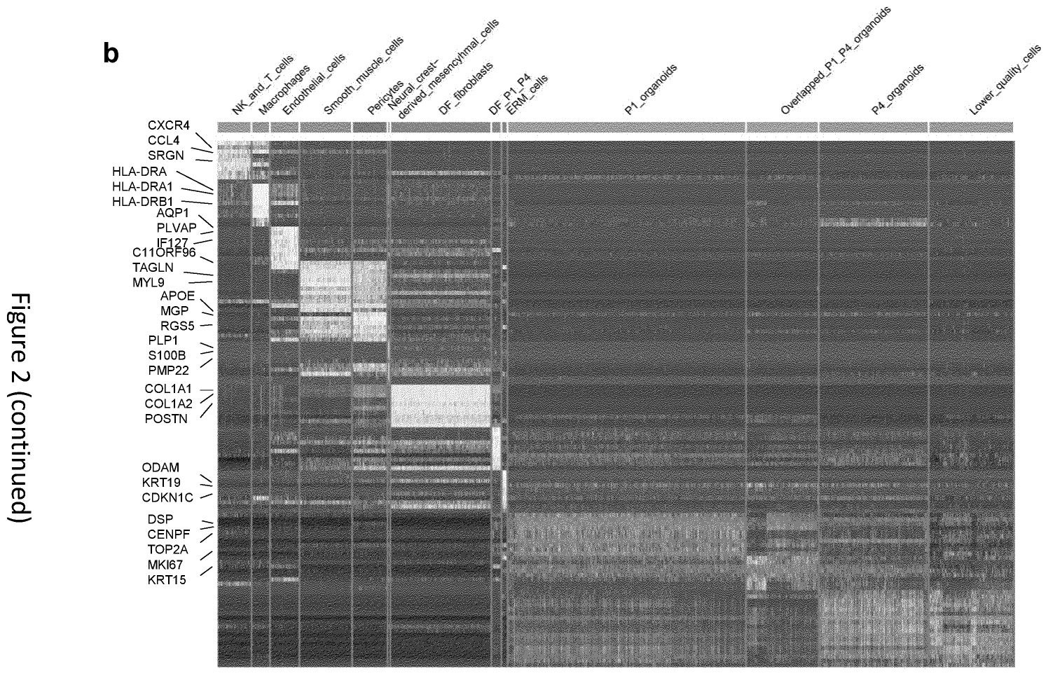

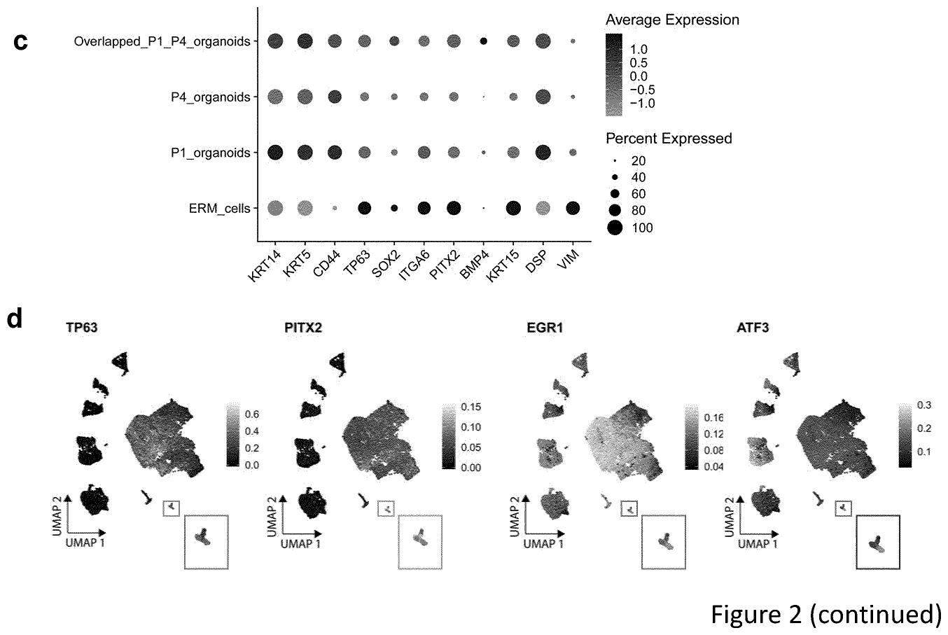

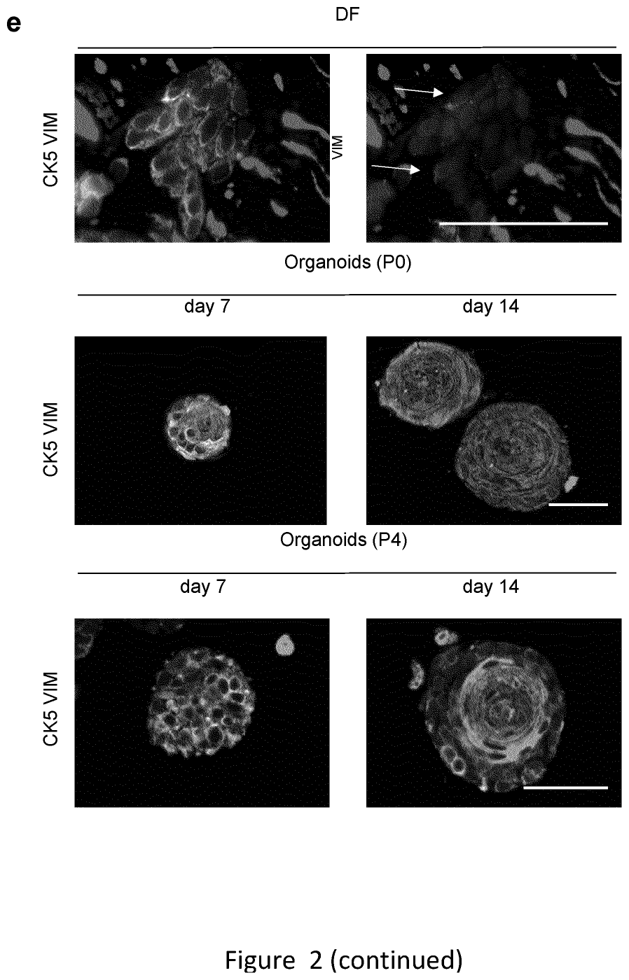

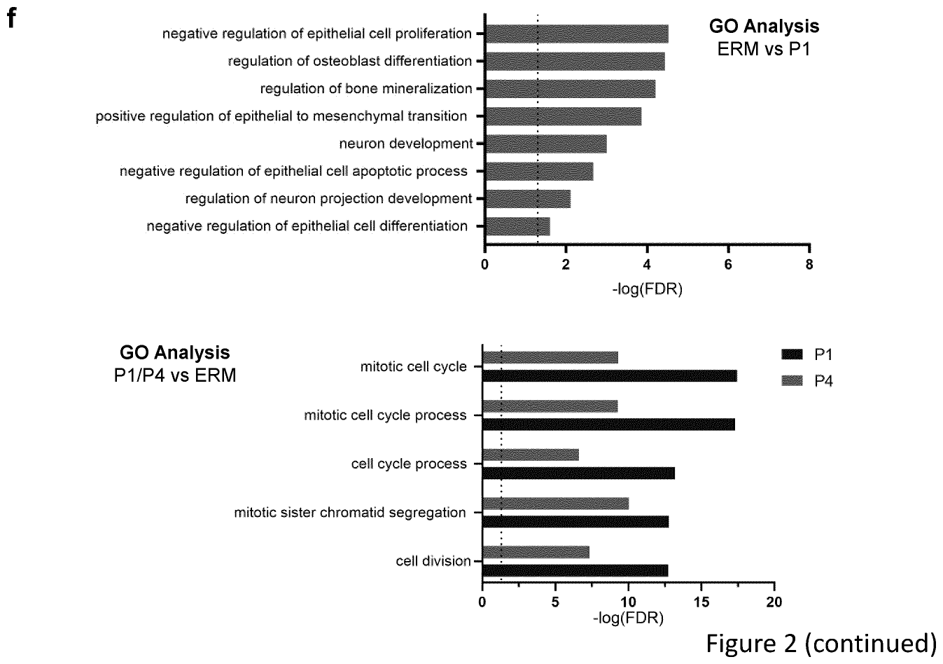

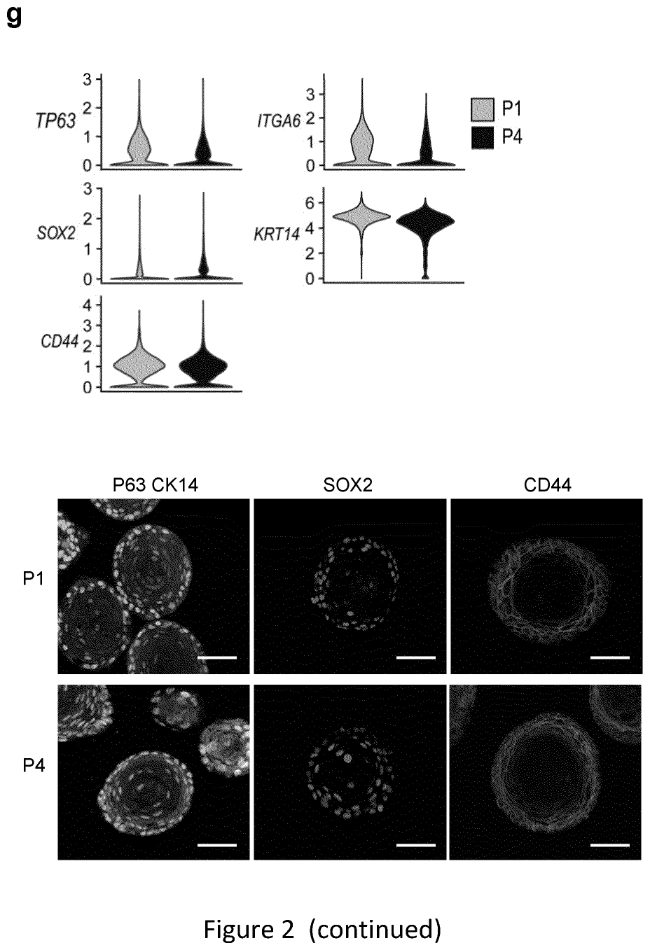

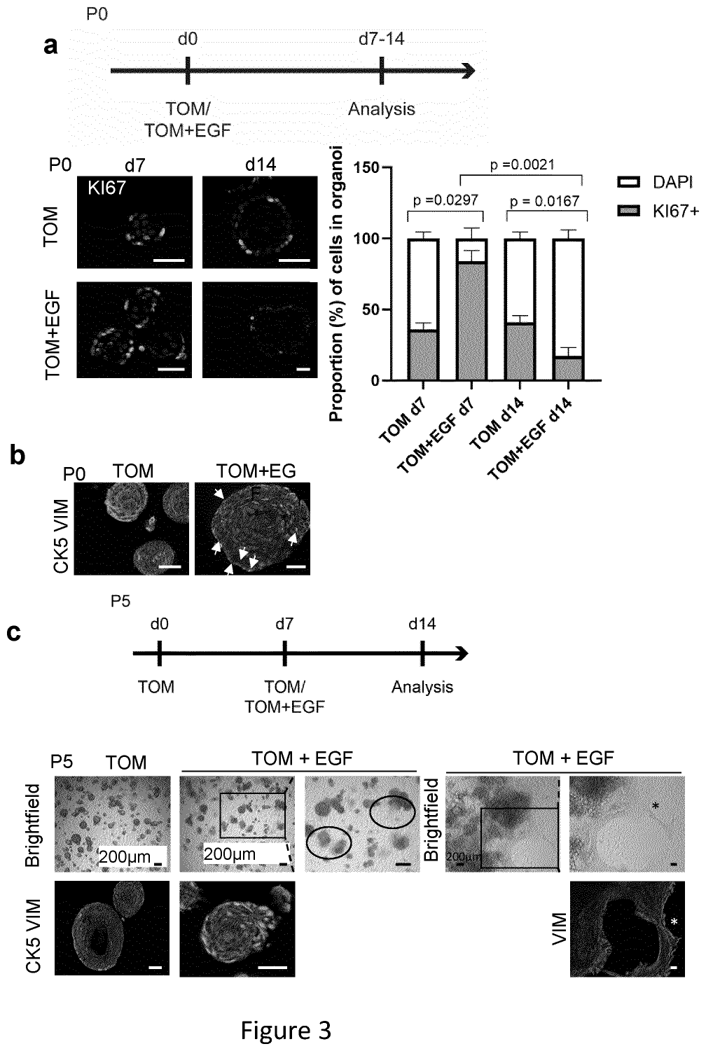

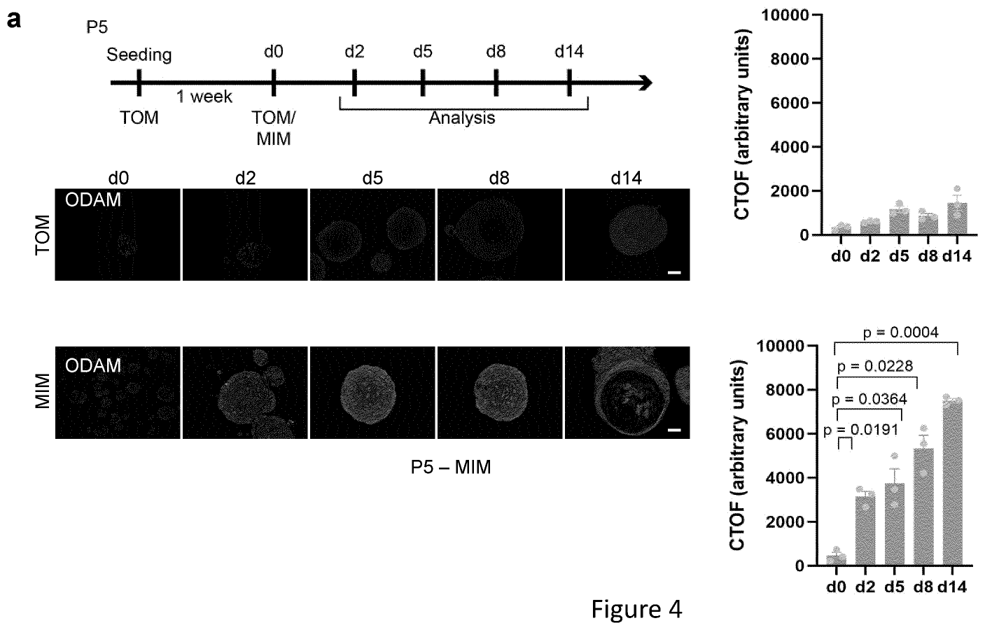

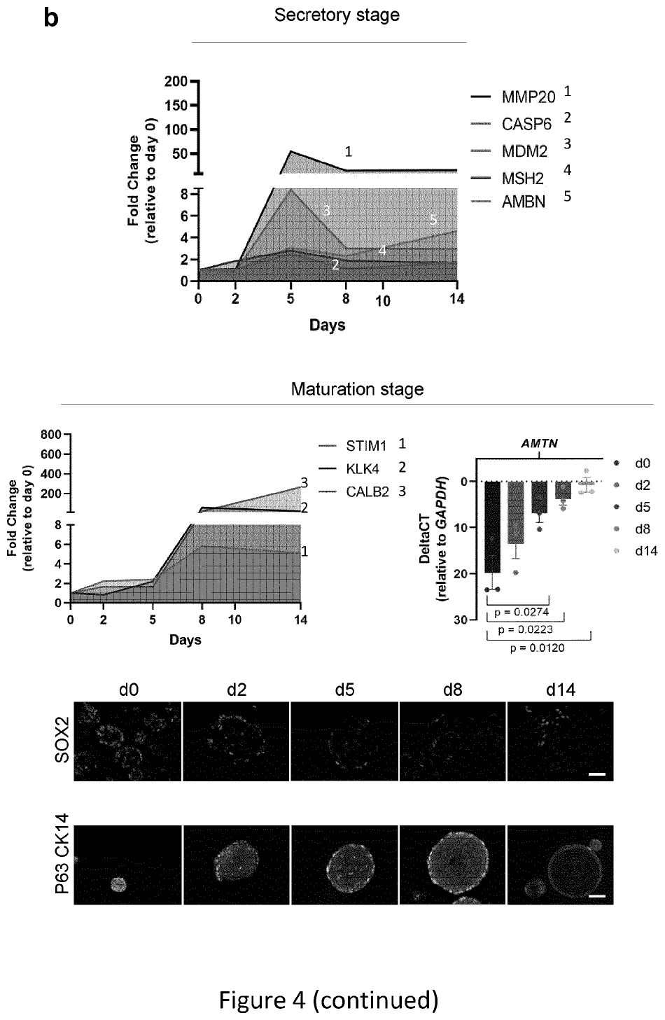

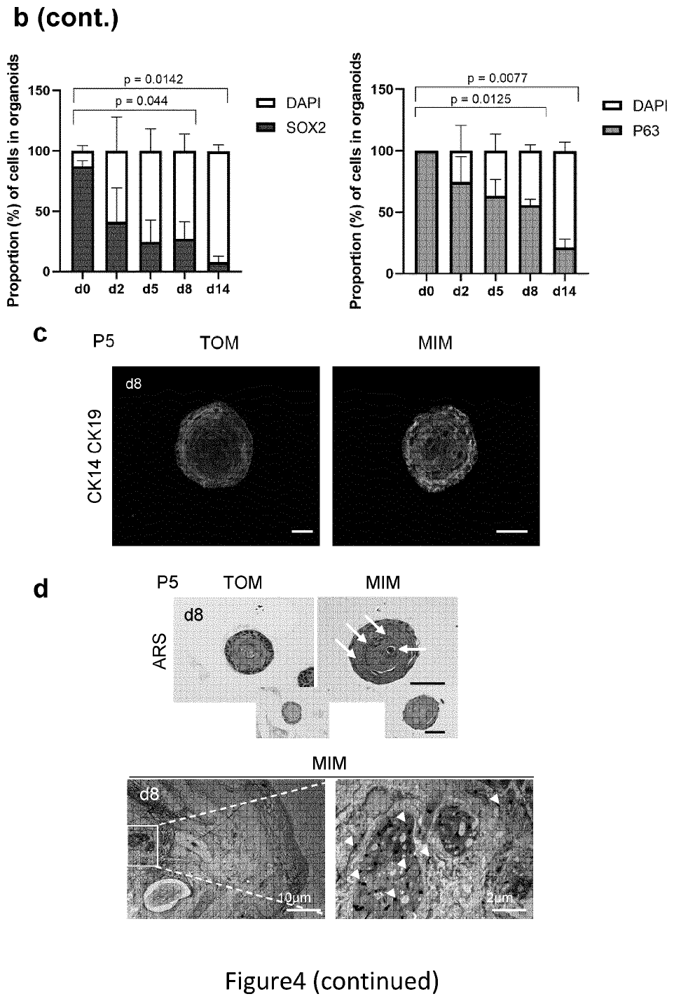

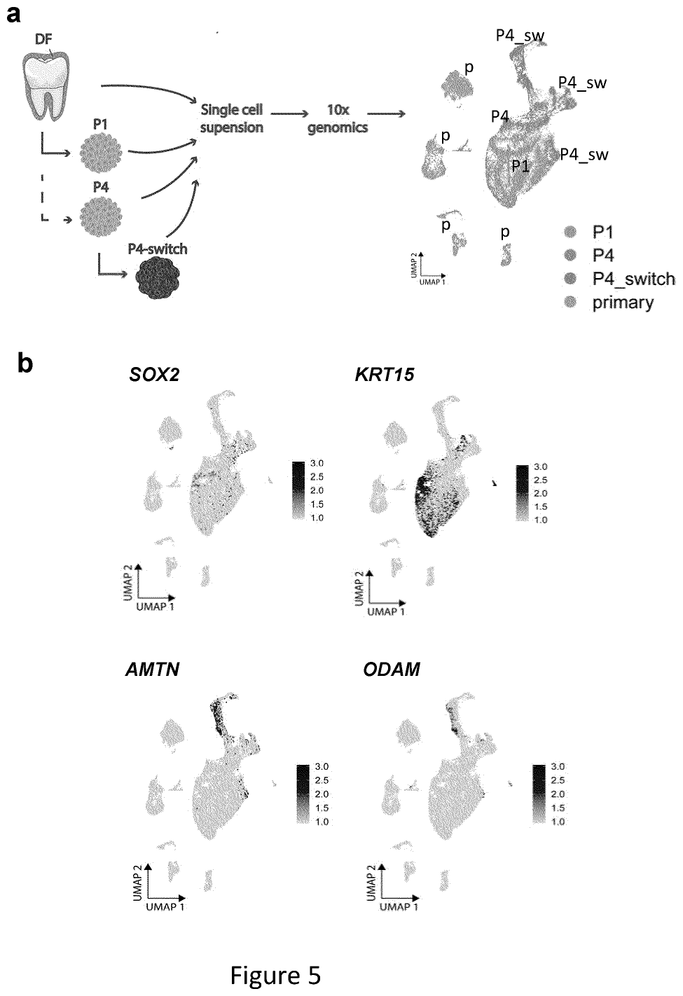

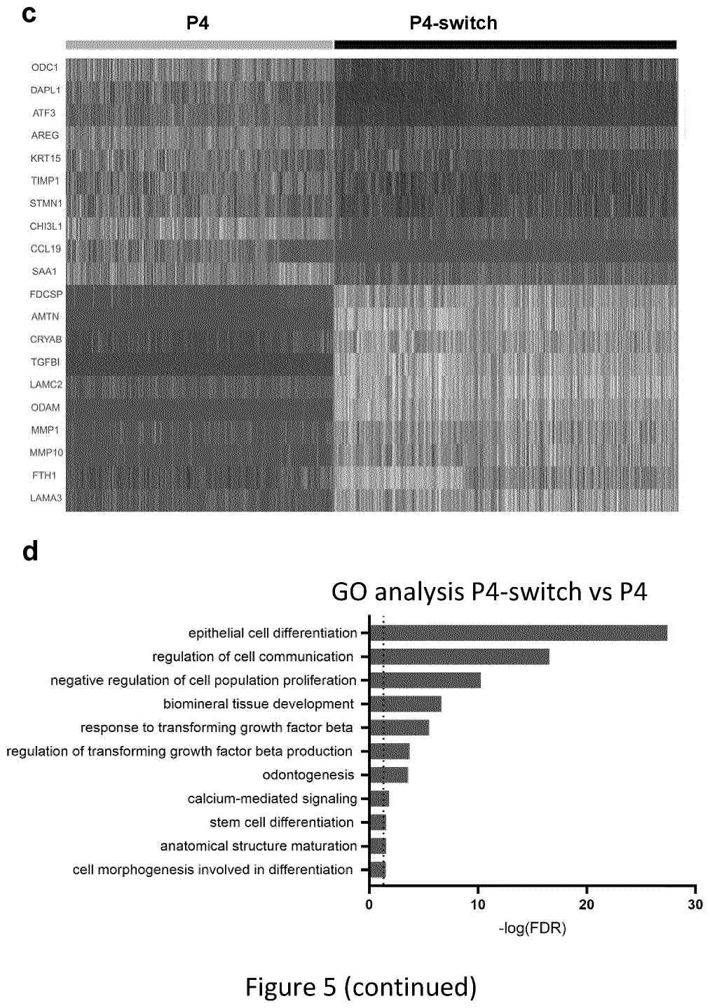

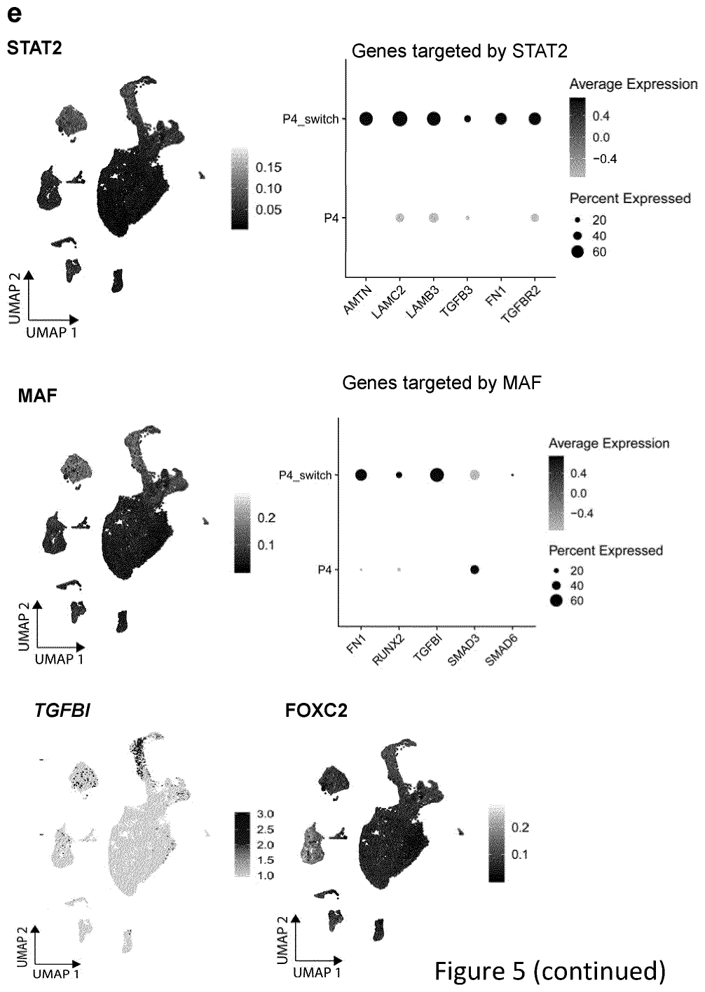

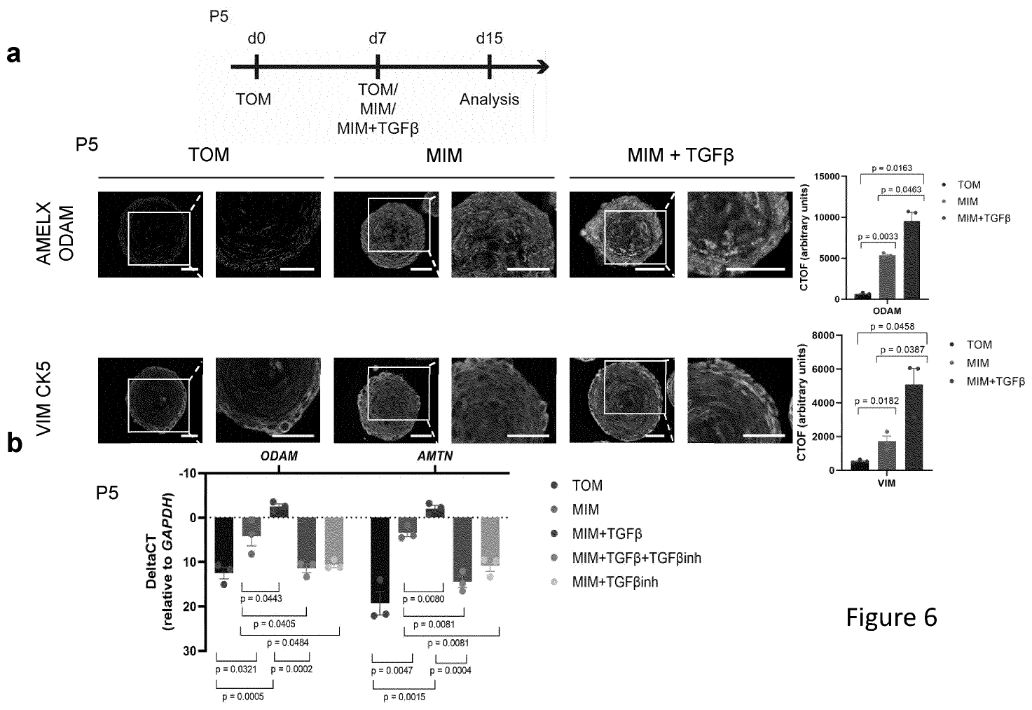

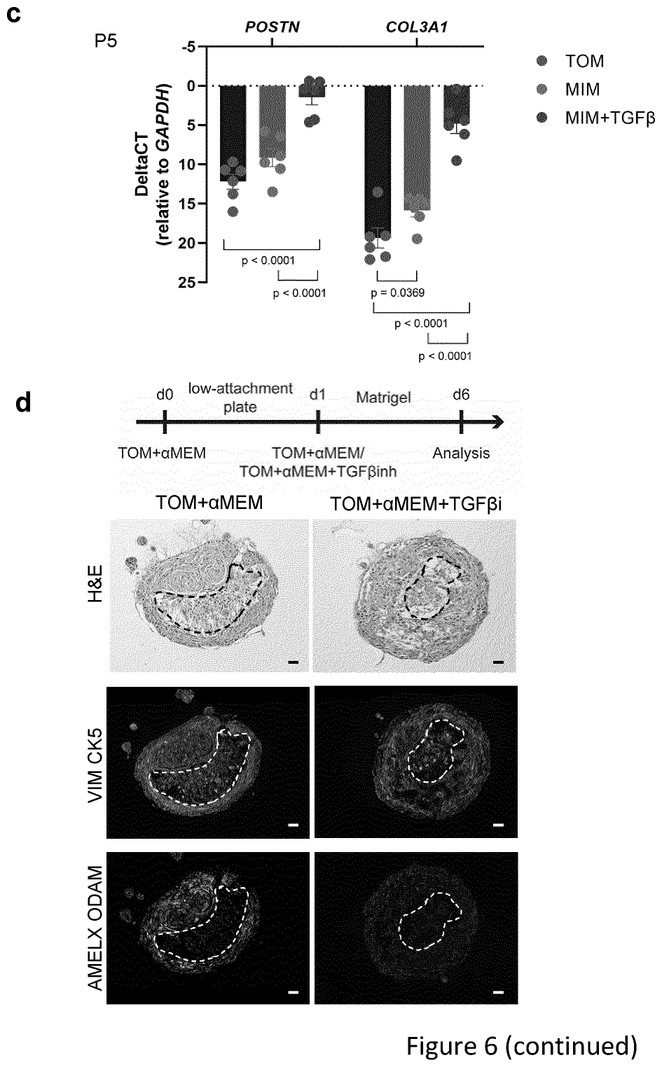

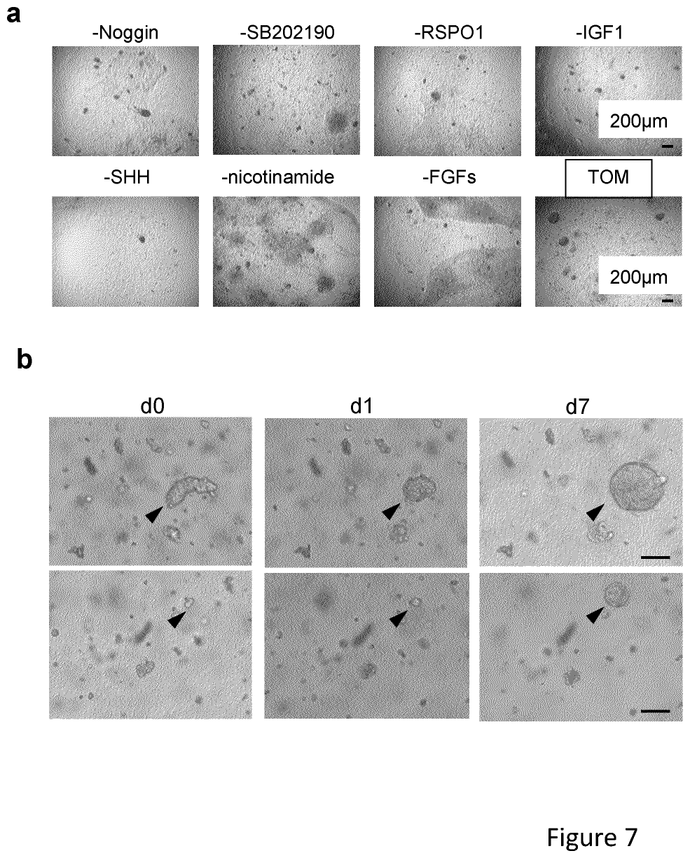

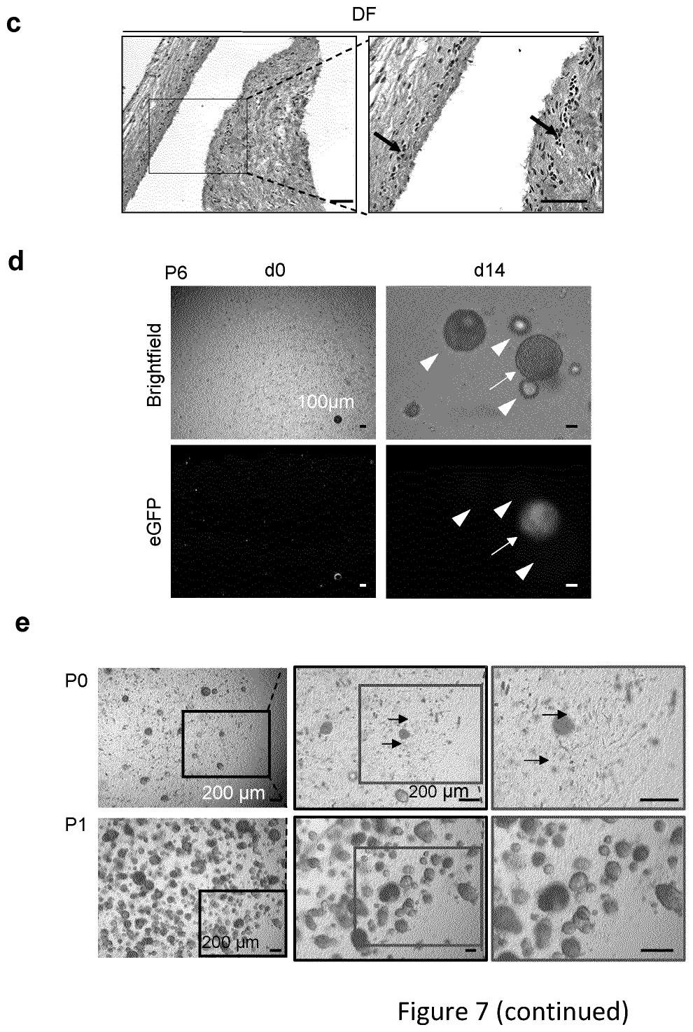

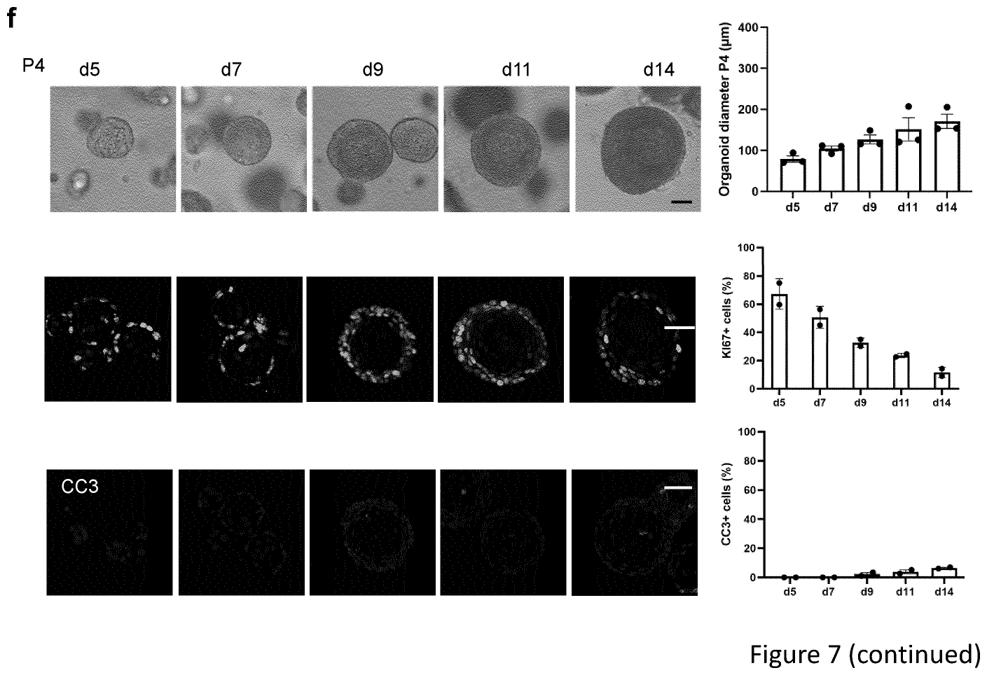

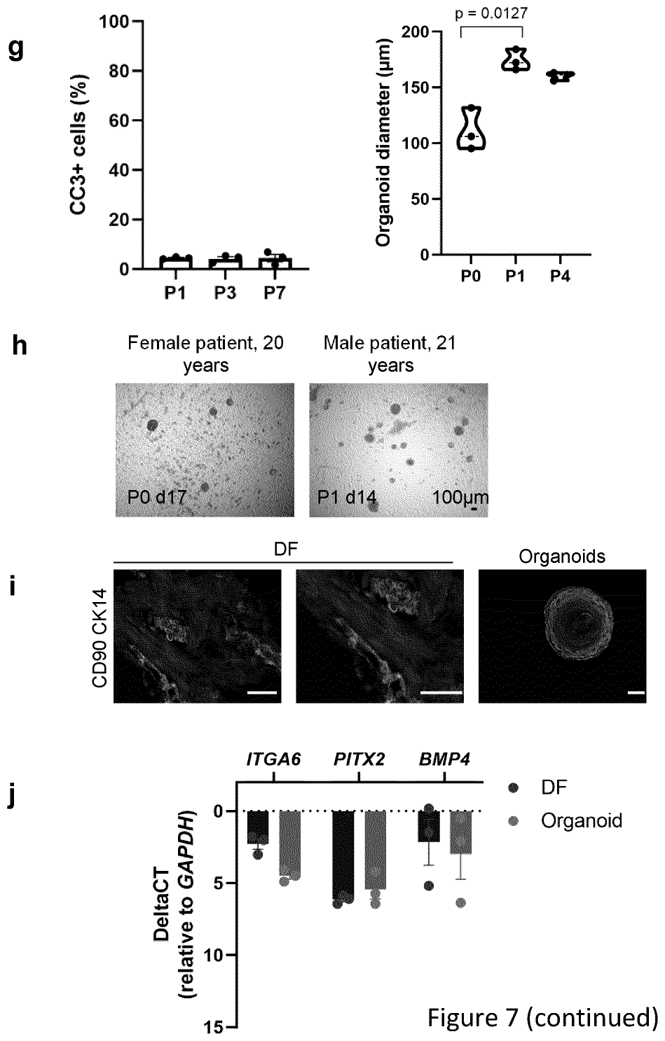

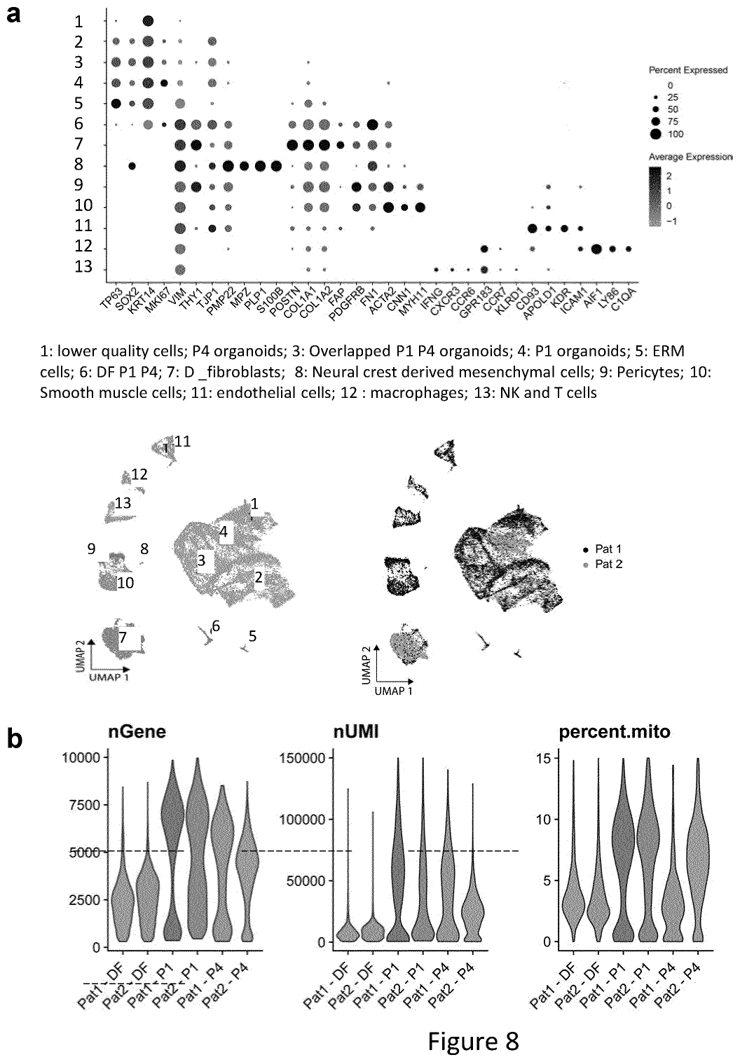

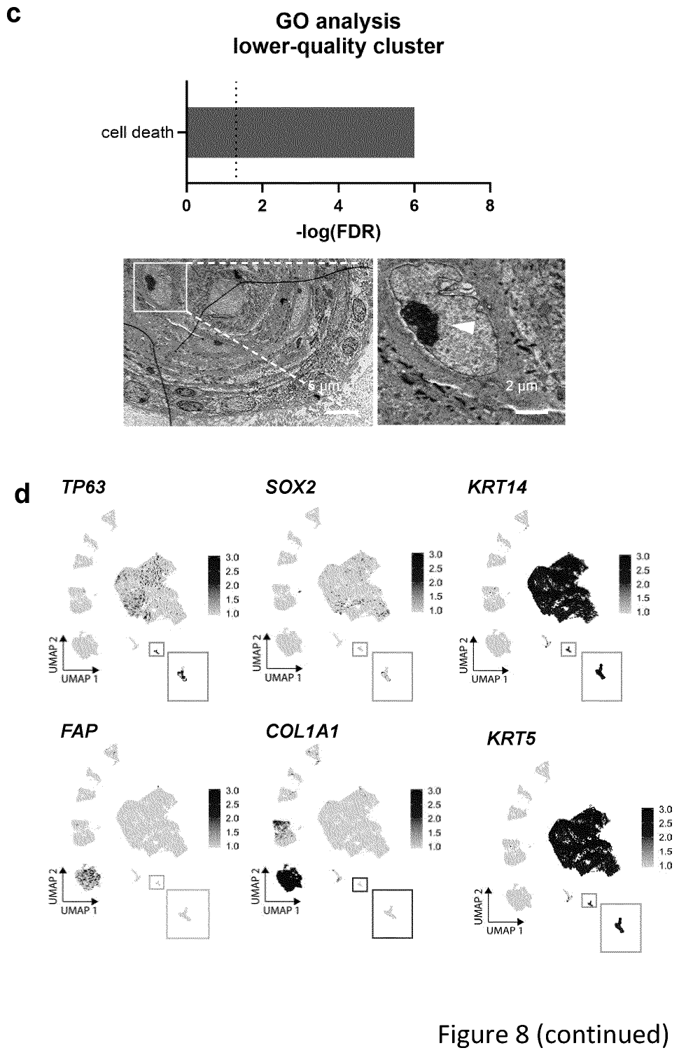

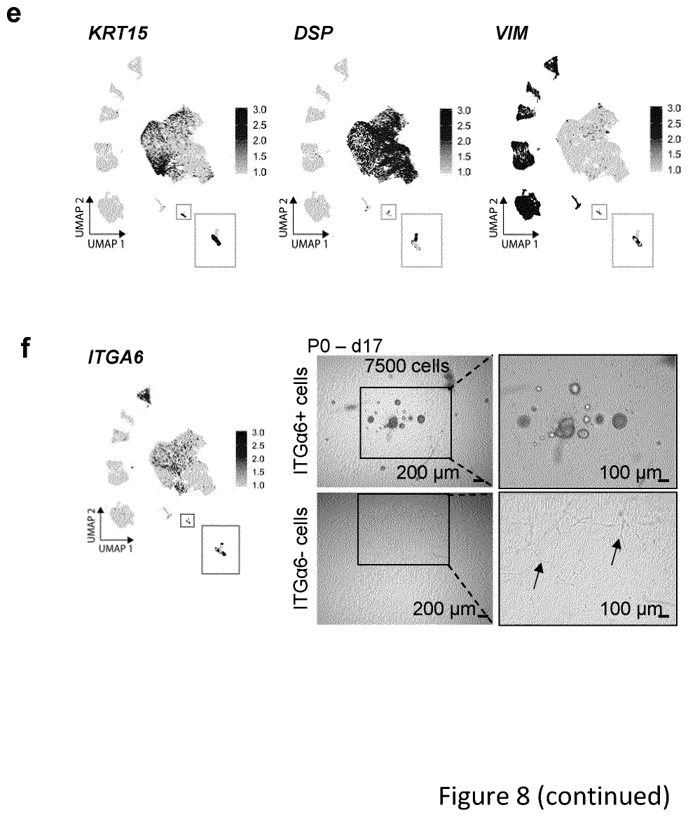

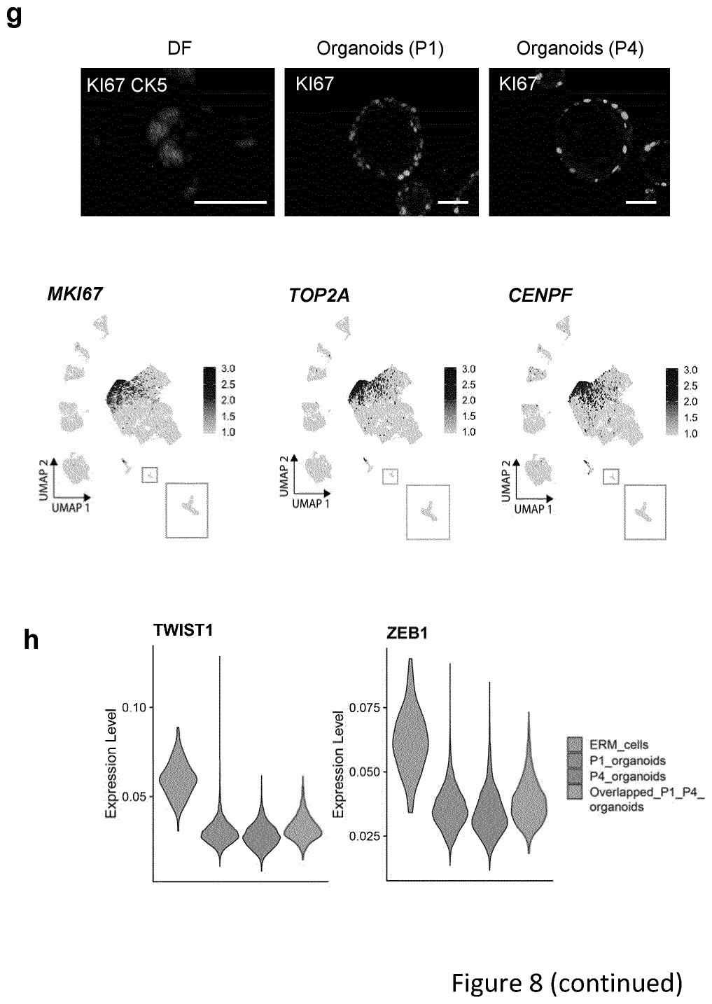

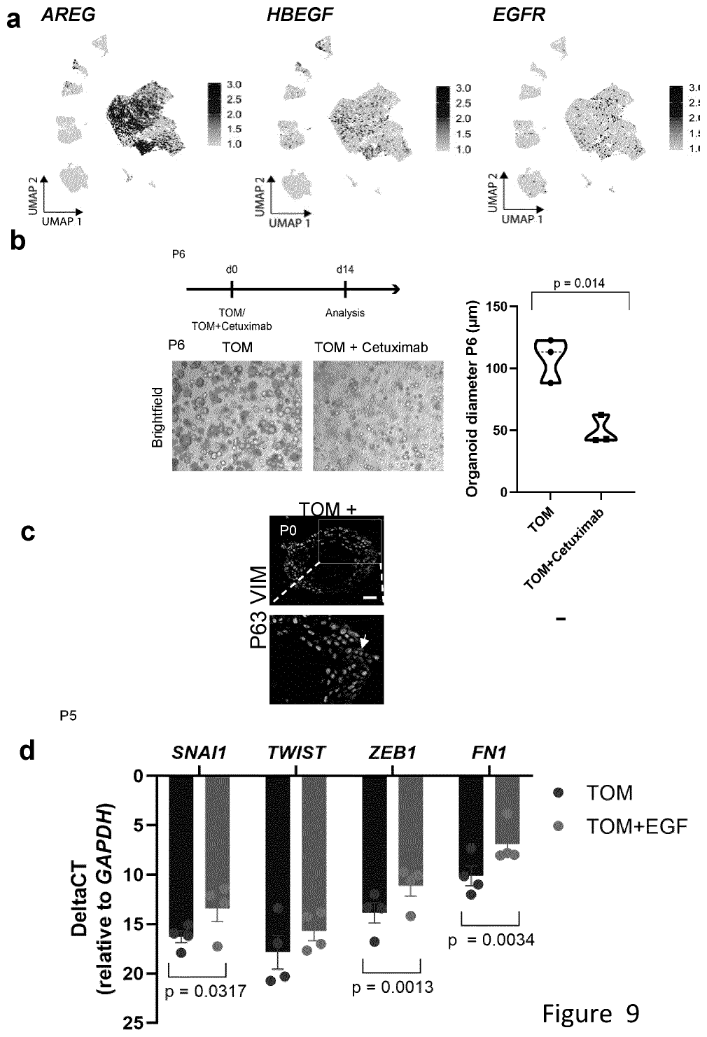

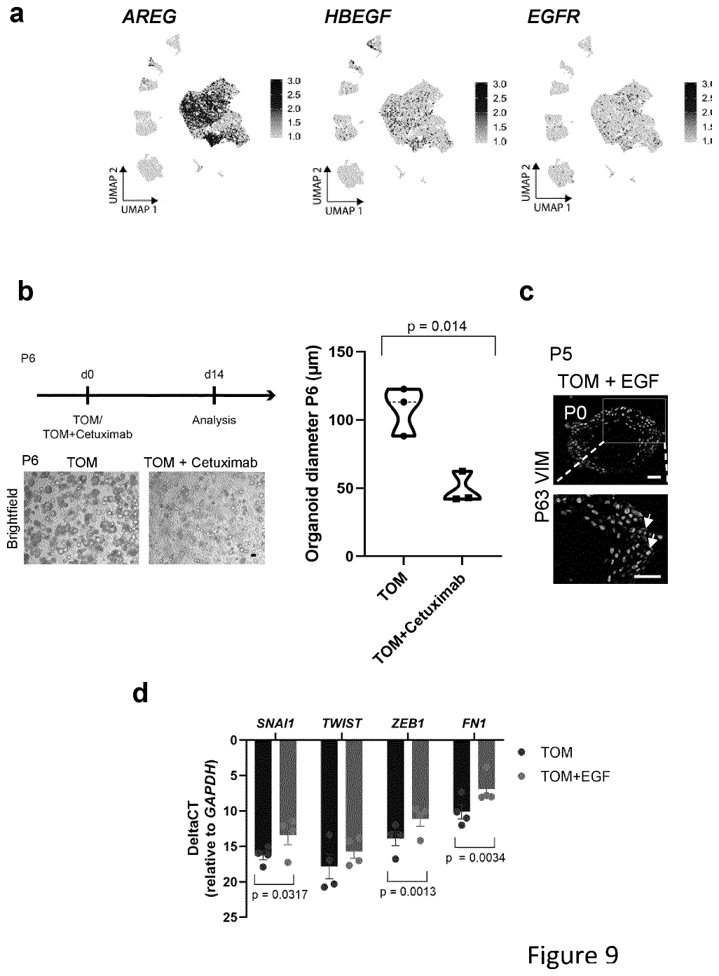

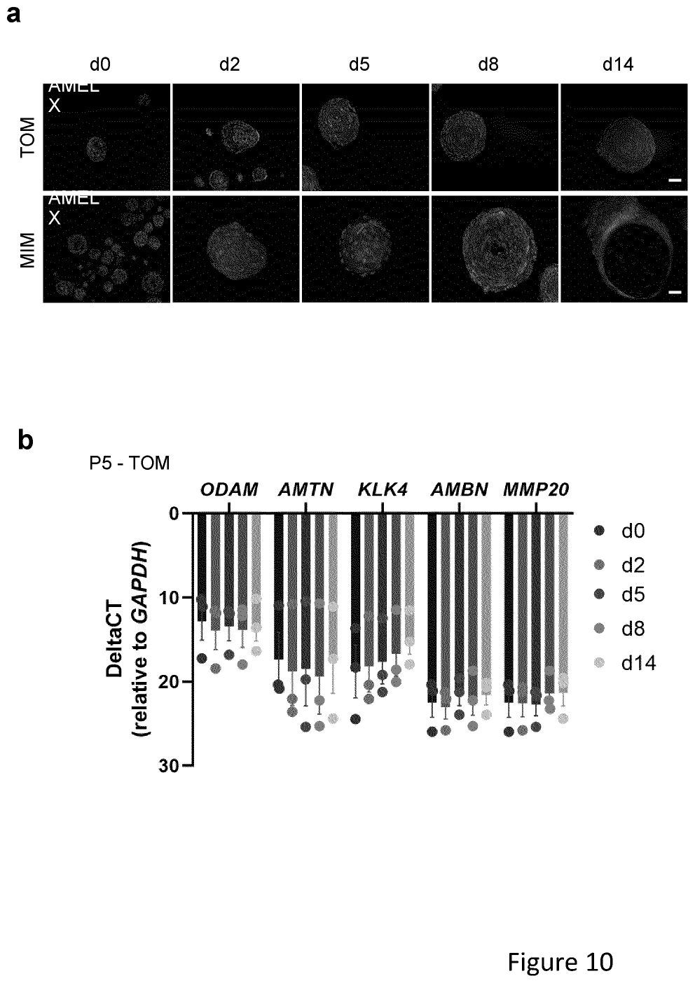

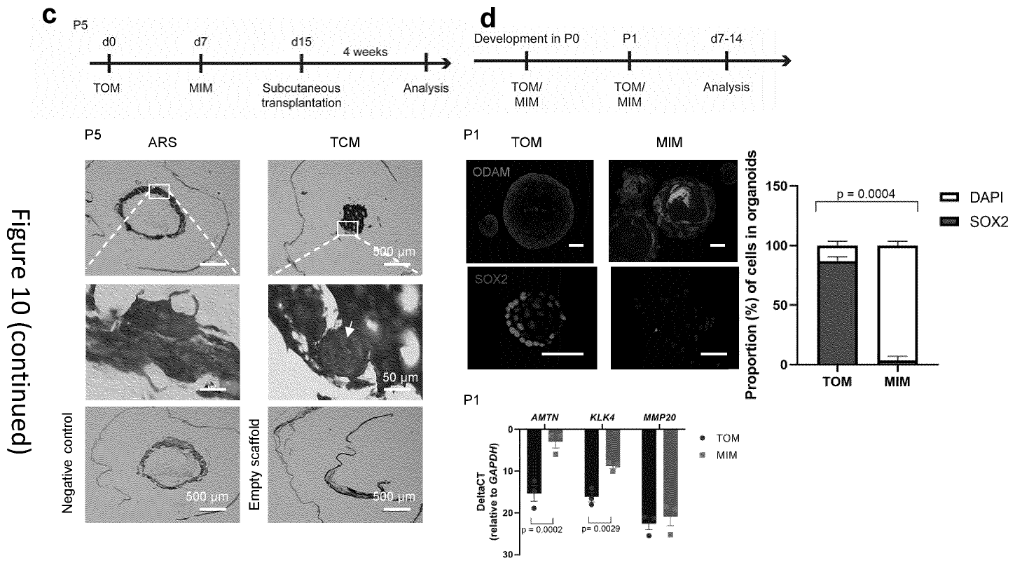

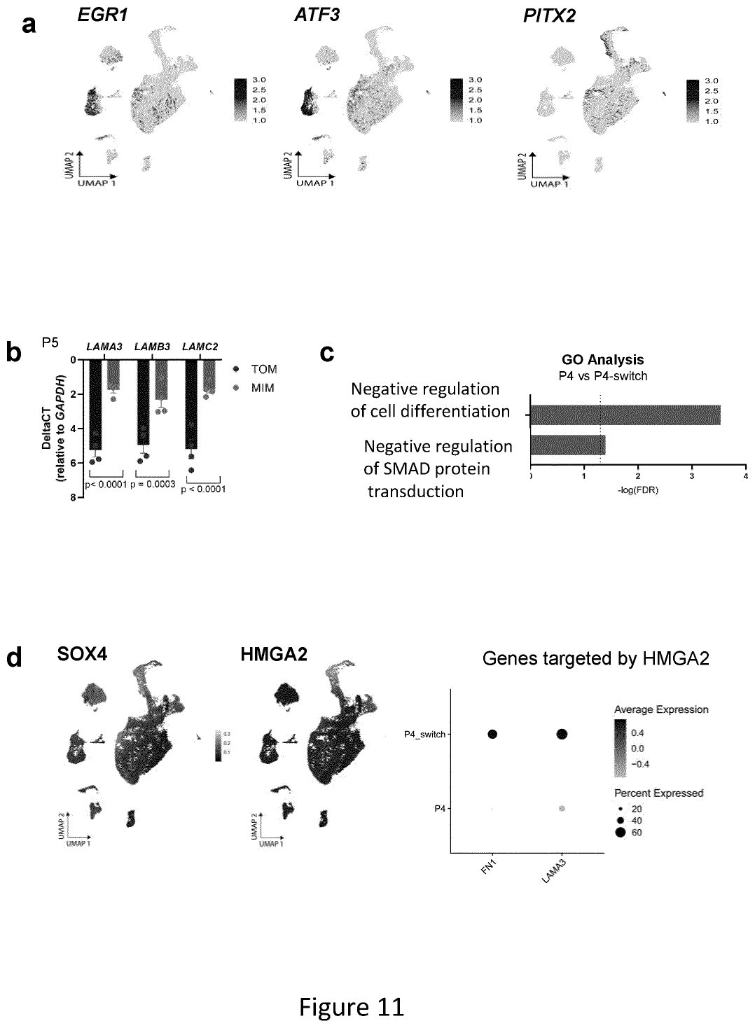

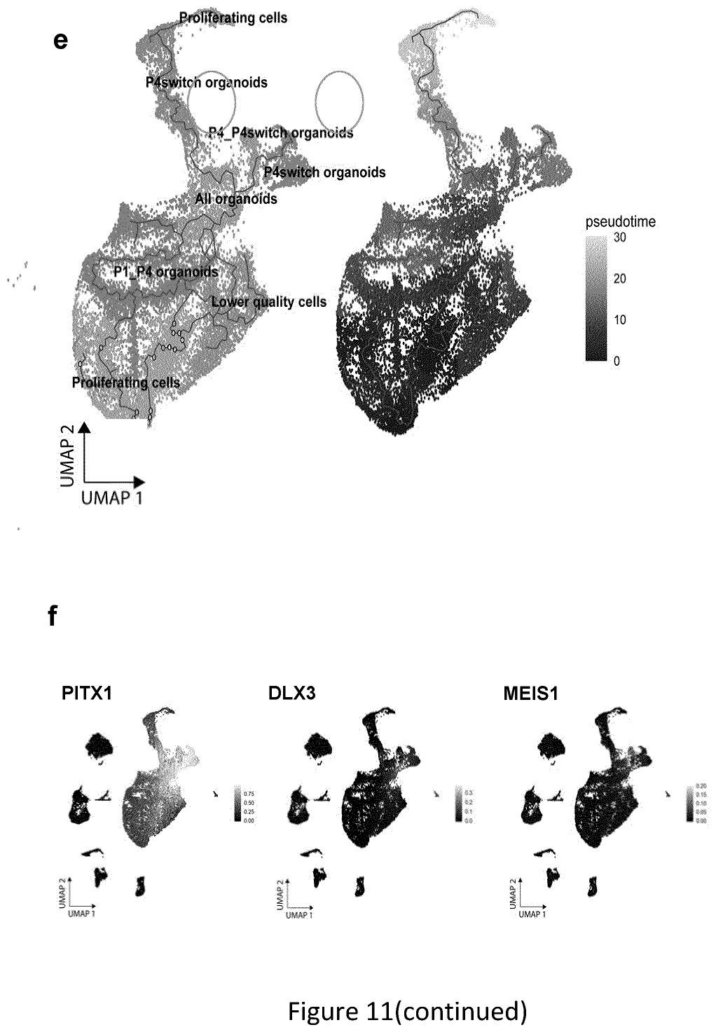

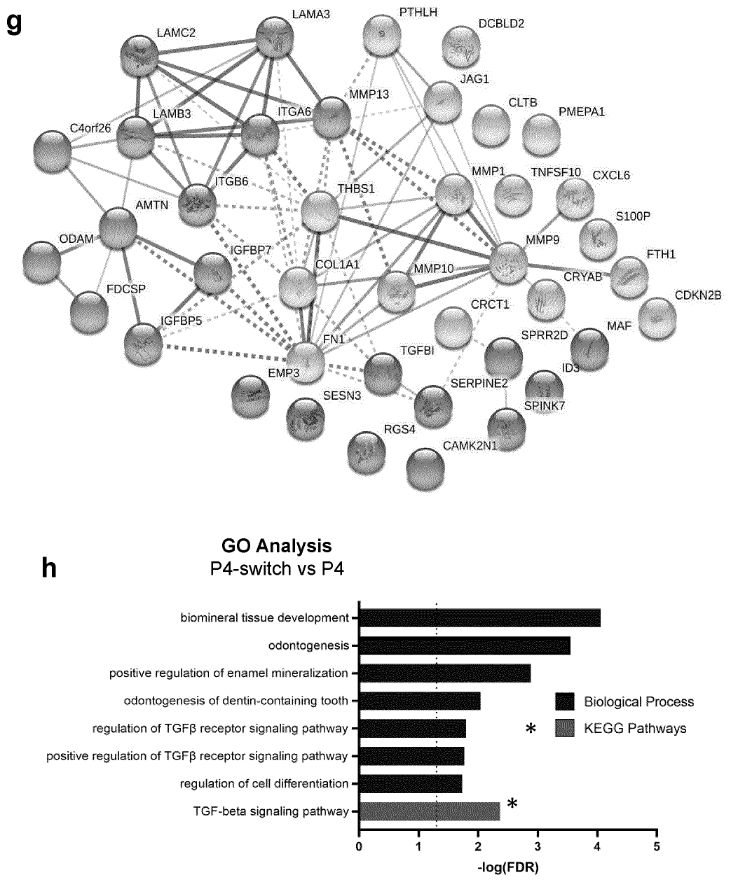

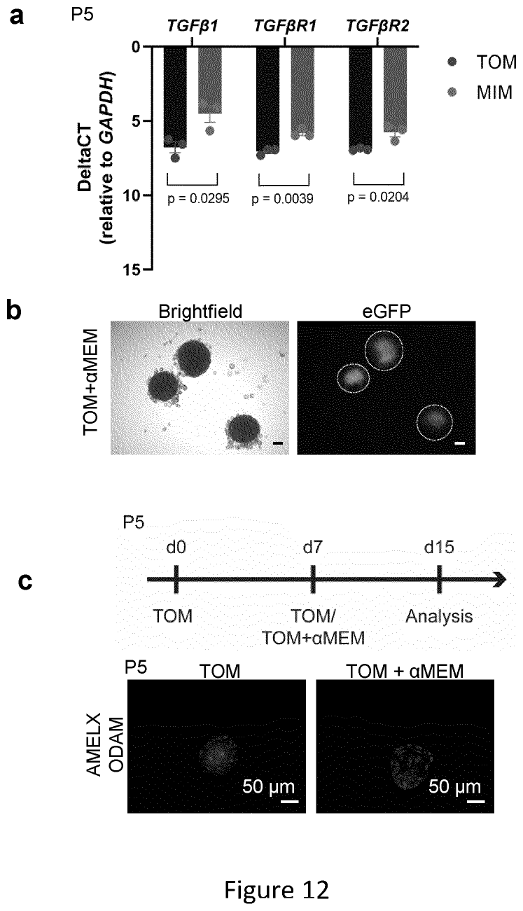

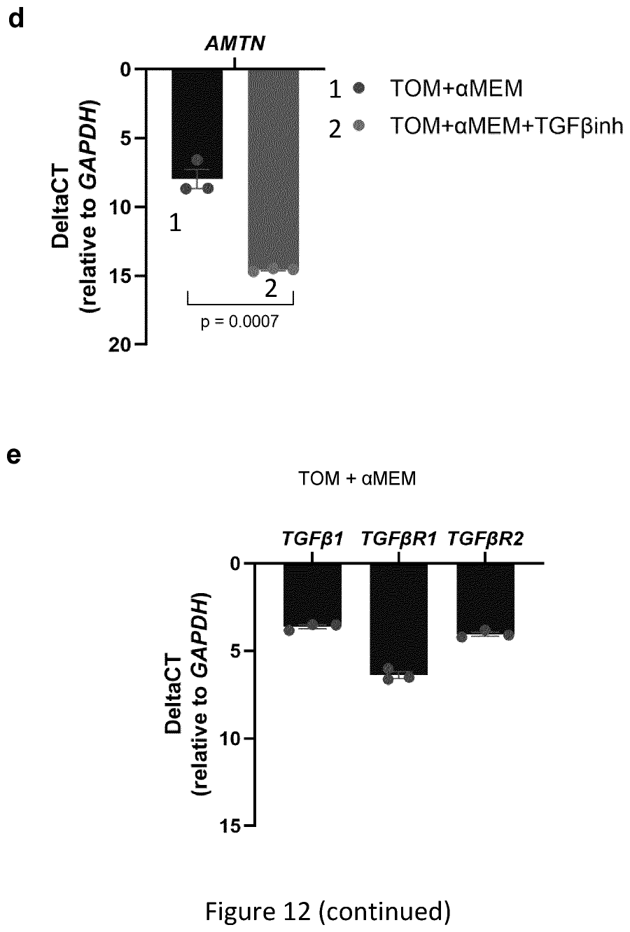

To develop epithelial organoids starting from human tooth, the dental follicle, known to encompass a large mesenchymal component but also the small epithelial epithelial cell rests of Malassez compartment, was isolated from unerupted third molars (wisdom teeth) extracted from adolescent patients (FIG. 1a). After tissue dissociation, the epithelial-mesenchymal cell mixture, comprising single cells and cell clusters, was embedded in Matrigel and cultured in a precisely defined medium. Organoids are typically established using a cocktail of growth and regulatory factors active in the tissue's epithelial stem cell niche. In case niche signals are unresolved, factors with a key role in the tissue's embryonic development are applied. Hence, since the human tooth epithelial stem cell niche is undefined, growth and signalling factors shown to play a role in tooth development were tested, including sonic hedgehog (SHH), fibroblast growth factors (FGFs) and insulin-like growth factor-1 (IGF1). The starting point is a medium containing these factors as well as generic organoid growth factors (such as WNT activators, nicotinamide, BMP inhibitor and p38 MAPK inhibitor), and assessed the essentiality of individual factors by omitting. The BMP inhibitor (Noggin), p38 MAPK inhibitor (SB202190), WNT activator (R-spondin 1 (RSPO1)), IGF1, SHH, nicotinamide, and FGFs (FGF2, FGF8, FGF10) were all evidently needed for efficient organoid formation (FIG. 7a). Eventually, this systematic evaluation led to defining an optimized medium (further referred to as tooth organoid medium or TOM; Table 1), allowing to develop organoid lines from dental follicle samples at 100% efficiency (Table 2).

TABLE 1
Tooth organoid medium (TOM)

| | | | | Catalogue |
|---|---|---|---|---|
| Product | Concentration | | Supplier | Number |
| Serum-free | | | Thermo Fisher | For composition, |
| defined medium | | | Scientific | see Table 4 |
| A83-01 | 0.5 | μM | Sigma-Aldrich | SML0788 |
| B27 (without | 2% | | Gibco | 12587-010 |
| vitamin A) | | | | |
| Cholera Toxin | 100 | ng/ml | Sigma-Aldrich | c8052-.5 mg |
| FGF10 | 100 | ng/ml | Peprotech | 100-26 |
| FGF2 (=basic FGF) | 20 | ng/ml | R&D Systems | 234-FSE-025 |
| FGF8 | 200 | ng/ml | Peprotech | AF-100-25 |
| L-glutamine | 2 | mM | Gibco | 25030024 |
| IGF-1 | 100 | ng/ml | Peprotech | 100-11 |
| N2 | 1% | | Gibco | 17502-048 |
| N-acetyl L-cysteine | 1.25 | mM | Sigma-Aldrich | A7250 |
| Nicotinamide | 10 | mM | Sigma-Aldrich | N0636 |
| Noggin | 100 | ng/ml | Peprotech | 120-10C |
| RSPO1 | 200 | ng/ml | Peprotech | 120-38 |
| SB202190 | 10 | μM | Biotechne | 1264 |
| | | | (Tocris) | |
| SHH | 100 | ng/ml | R&D Systems | 464-SH-200 |
| WNT3A | 200 | ng/ml | R&D Systems | 5036-WN-500 |

TABLE 2
Overview of patient-derived organoid lines

| Patient-derived | Age | |
|---|---|---|
| organoid line | (year) | Sex |
| Organoid_1 | 18 | Female |
| Organoid_2 | 15 | Female |
| Organoid_3 | 16 | Female |
| Organoid_4^{a} | 18 | Male |
| Organoid_5 | 19 | Male |
| Organoid_6 | 18 | Female |
| Organoid_7^{a} | 15 | Female |
| Organoid_8 | 16 | Male |
| Organoid_9 | 16 | Female |
| Organoid_10 | 17 | Male |
| Organoid_11 | 14 | Female |
| Organoid_12 | 15 | Male |
| Organoid_13 | 15 | Female |
| Organoid_14 | 17 | Female |
| Organoid_15 | 15 | Female |
| Organoid_16 | 17 | Female |
| Organoid_17 | 15 | Male |
| Organoid_18 | 15 | Female |
| Organoid_19 | 19 | Female |
| Organoid_20^{b} | 15 | Male |
| Organoid_21 | 17 | Male |
| Organoid_22^{b} | 18 | Female |
| Organoid_23 | 15 | Female |
| Organoid_24 | 15 | Female |
| Organoid_25 | 19 | Male |
| Organoid_26 | 17 | Male |
| Organoid_27 | 17 | Male |
| Organoid_28 | 17 | Male |
| Organoid_29 | 17 | Female |
| Organoid_30 | 15 | Female |

^{a}For in vivo transplantation
^{b}For scRNA-seq analysi

Organoid structures gradually developed in two weeks time (FIG. 1a; passage 0 (P0)), growing out of cell clusters (typically 4-8 cells wide by 20 cells long, similar to the epithelial cell rests of Malassez cell groups present in the primary dental follicle tissue; FIG. 7b,c) or of single cells, indicating the capability of clonal development (FIG. 7b). Importantly, the organoids were amenable to long-term expansion, at present for more than 10 consecutive passages (i.e. 5 months) (FIG. 1a). At passaging, the organoids were dissociated into single cells and organoid structures efficiently re-grew. When starting from a mixture of cells dissociated from either eGFP⁺ or eGFP⁻ organoid cultures, the organoids that reformed were homogeneously fluorescent or non-fluorescent, suggesting clonal regrowth at passaging (FIG. 7d). Mesenchymal cells, also present in the dissociated dental follicle cell mixture, adhered to the bottom of the culture plate following sample seeding (P0; FIG. 7e), and were swiftly lost at passaging in the standard, epithelial-favouring organoid culture conditions used (P1; FIG. 7e). During a single-passage 14-day culture period, organoids progressively increased in size while the proportion of proliferating (KI67+) cells gradually decreased and the fraction of apoptotic (cleaved-caspase 3, CC3+) cells slightly enhanced, although to only low levels which remained invariable over different passages (as determined in full-grown day-14 organoids) (FIG. 7f,g). Full-grown organoid size also remained constant over passaging, after a first significant increase from P0 to P1 (FIG. 7g). Within individual passages, the organoids displayed considerable size homogeneity (FIG. 7g). Finally, organoid cultures could be reconstituted after cryopreservation, and were also establishable from the dental follicle of already erupted wisdom teeth (FIG. 7h).

The developed organoid structures displayed a dense morphology (FIG. 1a,b), showing an outer border of stratified cuboidal epithelium (CE) with cells displaying a high nucleo-cytoplasmic ratio, and an adjoining stratified squamous epithelium (SE; FIG. 1b). In dental follicle tissue, epithelial cells with high nucleo-cytoplasmic ratio are present in the epithelial cell rests of Malassez (FIG. 7c). In further analogy, the epithelial cell rests of Malassez markers cytokeratin (CK) 14 and CK5 were detected in the organoids, as they are observed in compartments of the original dental follicle tissue (FIG. 1c). Importantly, the mesenchymal (fibroblast) marker CD90 (Thy-1 cell surface antigen, THY1) which is observed in compartments of the original dental follicle tissue was not detected in the organoids (FIG. 7i), indicating the absence of pure mesenchymal cells in the (epithelial) organoids. Previous studies proposed that the epithelial cell rests of Malassez contains dental epithelial stem cells, among others marked by CD44 and P63. Interestingly, these markers, indeed observed in the primary dental follicle tissue (FIG. 1d), were also detected in the derived organoids, both at initial formation (P0) and after passaging (P1; FIG. 1d). Moreover, the organoids and the native dental follicle tissue expressed SOX2 (FIG. 1d), a well-known marker of dental epithelial stem cells in mouse and detected in epithelium of developing teeth in humans [Yu & Klein (2020) Development 147, Dev184754]. Finally, the organoids showed prominent gene expression of the proposed epithelial cell rests of Malassez stem cell marker integrin-α6 (ITGA6), as well as of factors playing an important role in embryonic development of the dental epithelium (i.e. paired like homeodomain 2 (PITX2) and bone-morphogenetic protein 4 (BMP4)), all markers also detected in the original dental follicle tissue (FIG. 7j). Finally, expression of amelogenin (AMELX) was observed in both primary tissue (more in particular in the CK5⁺ epithelial cell rests of Malassez region of the dental follicle) and derived organoids (FIG. 1e). AMELX is not only a main constituent of enamel matrix but also a suggested marker of epithelial cell rests of Malassez cells (described in rat). Proposed functions of this epithelial cell rests of Malassez-produced AMELX include maintenance of periodontal ligament space and stimulation of cementoblast differentiation. Of note, AMELX localization can show a punctuated pattern, being present in vesicles and/or secreted in the extracellular space, which is also observed here (FIG. 1e).

### EXAMPLE 2. SINGLE-CELL TRANSCRIPTOMICS REINFORCES THE ORGANOID-EPITHELIAL CELL RESTS OF MALASSEZ CONGRUENCE

To decode the organoids in more granular detail, single-cell RNA-sequencing (scRNA-seq) analysis w applied on dental follicle-derived organoids (at P1 and P4) together with their primary tissue (FIG. 2a; Table 2). Unsupervised clustering of the aggregate data and visualization using Uniform Manifold Approximation and Projection (UMAP) exposed main dental follicle cell-type clusters annotated using reported markers comprising immune, endothelial, mesenchymal and epithelial compartments, and grouped organoid clusters with noticeable overlap of the P1 and P4 cultures (FIG. 2a; FIG. 8a). Of note, a cluster of lower quality cells (i.e. with low gene counts) was also discerned (FIG. 2a), not filtered out using the applied quality thresholds (see Methods; FIG. 8b). This cluster likely comprises dying cells from the organoids' core, as supported by Gene Ontology (GO) analysis revealing an enriched ‘cell death’ biological term (FIG. 8c), which conforms to ultrastructural features of apoptotic nuclei and absence of cell organelles in the organoids' centre (FIG. 8c).

Compiling the clusters' top 10 differentially expressed genes (DEGs) in a heatmap exposed specific expression patterns of the different clusters (FIG. 2b). Several of the reported stem cell and epithelial cell rests of Malassez markers were found expressed in the organoids (FIG. 2c; FIG. 8a,d), thus corroborating the observations above on organoid-epithelial cell rests of Malassez correspondence. Of note, mesenchymal markers such as fibroblast activation protein alpha (FAP) and collagen type I alpha 1 chain (COL1A1), being present in the dental follicle mesenchymal (fibroblast) cluster, were not detected in the organoid clusters, thereby again demonstrating the absence of pure mesenchymal cells in the (epithelial) organoids (FIG. 8d). In further analogy with the transcriptomic organoid-epithelial cell rests of Malassez congruence, gene-regulatory network (regulon) analysis using pySCENIC (i.e. defining core transcription factors with their positively regulated target genes in single cells) showed high TP63 and PITX2 regulon activity in both organoids and epithelial cell rests of Malassez (FIG. 2d). Predicted target genes of the PTIX2 regulon include SOX2, TP63, PITX2, KRT5, KRT14 and BMP4 (FIG. 2c). Interestingly, the newly proposed mouse incisor epithelial (stem) cell markers KRT15 and dentin sialoprotein (DSP) were among the top 10 DEGs in the organoid as well as epithelial cell rests of Malassez clusters (FIG. 2b,c; FIG. 8e). Moreover, high regulon activity of early growth response 1 (EGR1) (marking a transient progenitor population in mouse incisor) was observed in organoid clusters and epithelial cell rests of Malassez, while substantial regulon activity of activating transcription factor 3 (ATF3), reported as mouse incisor epithelial cell marker, also showed up in the organoid and epithelial cell rests of Malassez clusters (FIG. 2d). Together, this single-cell transcriptomic analysis uncovered new, mouse-mirroring molecular features for human epithelial cell rests of Malassez, at present ill-defined. Gene expression of the mesenchymal marker vimentin (VIM) was detected in epithelial cell rests of Malassez cells (FIG. 2c; FIG. 8a,e). Co-expression of VIM was observed in epithelial cell rests of Malassez (CK5⁺) cells of the primary dental follicle tissue (FIG. 2e), which was recapitulated in the initiating organoids (FIG. 2e; P0, day 7). These findings may point to a hybrid epithelial/mesenchymal (E/M) nature which has been correlated with stemness and active stem cells in several other (developing) tissues (such as human foetal pituitary, intestine, liver, lung). VIM expression faded during further organoid culturing (FIG. 2e; day 14). However, VIM expression transiently reiterated at each passaging (as shown for P4; FIG. 2e), suggesting that the hybrid E/M (active stem cell) phenotype is re-activated at re-seeding. Taken together, the present profound single-cell transcriptomic analysis further reinforces the congruence between the dental follicle-derived organoids and the epithelial cell rests of Malassez (stem) cells residing in this tooth tissue. To finally corroborate this relationship, epithelial cell rests of Malassez stem cells were isolated from dental follicle tissue by FACS based on ITGα6 expression (FIG. 8f), and the cells were seeded in organoid-developing conditions. The ITGα6⁺ epithelial cell rests of Malassez cells formed organoids whereas ITGα6⁻ cells did not (FIG. 8f). Interestingly, the latter culture only showed mesenchymal cells adhering to the bottom of the culture plate which were not observed in the ITGα6⁺ cell culture (FIG. 8f), further supporting that the dental follicle's mesenchymal cells do not contribute to, or make part of, organoid growth. The organoids were further compared with the epithelial cell rests of Malassez by applying GO analysis. Negative regulation of ‘epithelial cell proliferation’, of ‘epithelial cell apoptotic process’ and of ‘epithelial cell differentiation’ are significantly enriched in the epithelial cell rests of Malassez versus the organoids (P1) (FIG. 2f) which is in line with the quiescence stemness character of epithelial cell rests of Malassez under homeostatic conditions as reported in Davis (2018) J. Vet. Dent. 35, 290-298. For instance, the cell cycle inhibitor CDKN1C is among the top DEGs in the epithelial cell rests of Malassez cluster (FIG. 2b). On the other hand, GO terms associated with cell cycle division are enriched in the organoids when compared to epithelial cell rests of Malassez (FIG. 2f). In analogy, gene expression of the proliferation marker MKI67 is prominent in the organoid clusters and absent in the epithelial cell rests of Malassez cluster, further corroborated by immunostaining (FIG. 2b; FIG. 8g). Also, other proliferation markers such as topoisomerase II alpha (TOP2A) and centromere protein F (CENPF) were found almost exclusively expressed in the organoid clusters (FIG. 8g). Interestingly, CENPF was discovered in the dental epithelium of the continuously growing mouse incisor. Together, these data provide supportive evidence that the epithelial cell rests of Malassez stem cells, being quiescent in vivo, are proliferatively (re-) activated in organoid culture.

In further GO analysis, it was found that the biological terms ‘regulation of osteoblast differentiation’, ‘regulation of bone mineralization’ and ‘regulation of neuron projection development/neuron development’ are enriched in the epithelial cell rests of Malassez (FIG. 2f), all representing previously proposed biological functions of this specific dental follicle cell compartment [Davis (2018) J. Vet. Dent. 35, 290-298]. Finally, the higher mesenchymal character of the epithelial cell rests of Malassez as compared to full-grown organoids (FIG. 2c,e) is reflected in the enriched ‘positive regulation of epithelial-mesenchymal transition’ (EMT) term (FIG. 2f), in agreement with higher regulon activity of the EMT-driving transcription factors TWIST1 and ZEB1 in the epithelial cell rests of Malassez (FIG. 8h).

In general, gene expression signatures of P1 and P4 organoids display substantial similarity (FIG. 2b). In particular, expression of stemness markers remains comparable after the additional passaging (FIG. 2g), indicating that the tooth organoids retain their stemness phenotype during expansive culture.

### EXAMPLE 3. EGF INDUCES A PROLIFERATIVE AND EMT PHENOTYPE IN TOOTH ORGANOIDS, REMINISCENT OF IN VIVO EVENTS IN THE EPITHELIAL CELL RESTS OF MALASSEZ

To establish organoids from primary tissues, supplementation of EGF is generally found quintessential. Hence, it is remarkable that dental follicle-derived organoids develop and expand in the absence of exogeneous EGF (TOM; Table 1). scRNA-seq mining exposed that the EGF receptor (EGFR) ligands amphiregulin (AREG) and heparin-binding EGF (HB-EGF), together with EGFR, are highly expressed in the organoids (FIG. 9a) which may substitute for EGF. In line, blocking the EGFR with cetuximab (added at passaging) compromised organoid growth, resulting in smaller organoids (FIG. 9b), thereby revealing the need for endogenous EGFR signalling. In vivo, it is known that elevated EGF levels in the epithelial cell rests of Malassez compartment (as, for instance, occurring upon tooth movement, infection or trauma) activates epithelial cell rests of Malassez cell proliferation [Davis (2018) J. Vet. Dent. 35, 290-298]. Supplementation of EGF (50 ng/ml) to initiating organoid cultures (medium referred to as TOM+EGF) resulted in an increased number of proliferating (KI67⁺) cells in the organoids (P0; day 7; FIG. 3a). The increase was swiftly followed by a decline in proliferation (day 14; FIG. 3a), coinciding with the induction of an EMT process, as supported by the emergence of VIM expression in the organoids' border, found colocalized with epithelial CK5 or P63 in several cells (FIG. 3b; FIG. 9c). Moreover, exposing the organoids to EGF after preceding growth and expansion in TOM led to a same process with appearance of VIM⁺ cells (FIG. 3c), as well as migration of cells out of the organoid structures to grow at the bottom of the culture plate, displaying mesenchymal (spindle-like) morphology and VIM expression (FIG. 3c). This motile behaviour further underscores the occurrence of EMT, also supported by the upregulated expression of specific mesenchymal/EMT-linked factors (FIG. 9d). It has been proposed that EMT induction in the epithelial cell rests of Malassez (as, for instance, occurring upon damaging tooth impact) enables the cells to migrate and eventually contribute to regeneration of neighbouring tissues [Davis (2018) J. Vet. Dent. 35, 290-298].

### EXAMPLE 4. THE TOOTH ORGANOIDS ARE AMENABLE TO AN AMELOBLAST DIFFERENTIATION PROCESS

During tooth development, dental epithelial stem cells give rise to ameloblasts which produce enamel matrix proteins (EMPs) for amelogenesis [Yu & Klein (2020) Development 147, dev184754]. It has also been shown that epithelial cell rests of Malassez can differentiate into ameloblast (-like) cells [Hamamoto et al. (1996) Oral Surg. Oral Med. Oral Pathol. Oral Radiol. Endod. 81, 703-709] and produce EMPs. Ameloblast differentiation encompasses a secretory stage with production of the EMPs AMELX and ameloblastin (AMBN), and a maturation stage during which amelotin (AMTN) and odontogenic-ameloblast associated protein (ODAM) are produced. The EMPs are proteolytically cleaved by matrix metalloproteinase 20 (MMP20) and kallikrein (KLK4), typically expressed during the secretory and maturation phase, respectively. Here, it was examined whether the dental follicle-derived organoids, possessing an epithelial epithelial cell rests of Malassez-stemness phenotype, can be driven into differentiation toward ameloblasts.

Organoids expanded in TOM were switched to a medium previously reported to trigger ameloblast-like differentiation in 2D dental epithelial stem cell cultures [Yan et al. (2006) Eur. J. Oral Sci. 114, 154-158], (referred to as mineralization-inducing medium, MIM; Table 3), and analysed at multiple time points (FIG. 4a).

TABLE 3
Mineralization-inducing medium (MIM)

| | | | | Catalogue |
|---|---|---|---|---|
| Product | Concentration | | Supplier | Number |
| Keratinocyte | | | Lonza | CC-3103 |
| serum-free | | | | |
| medium (KBM-2) | | | | |
| Calcium | 0.09 | mM | Thermo Fisher | AC349610250 |
| | | | Scientific | |
| EGF | 1 | ng/ml | R&D systems | 236-EG-200 |
| Bovine | 50 | μg/ml | Thermo Fisher | 13028014 |

| | |
|---|---|
| pituitary extract | Scientific |

Interestingly, ODAM expression swiftly emerged (from day 2) in the organoids switched to MIM, and increased in intensity while remaining absent in TOM-cultured organoids (FIG. 4a). AMELX protein, being already detected in standard TOM conditions (FIG. 1e), became visually more abundant in MIM-switched organoids from day 8, while remaining more constant in TOM-cultured organoids (FIG. 10a). Time-lapse gene expression analysis showed that secretory-stage markers of amelogenesis, mainly increased at day 5 in MIM, while maturation-stage markers peaked at day 8-14 (FIG. 4b). In contrast, expression did not change in TOM-cultured organoids (FIG. 10b). In parallel with the differentiation process, the stemness phenotype of the organoids dropped showing a fast decline in SOX2⁺ and P63⁺ cells (FIG. 4b). Interestingly, CK19 expression emerged in MIM-switched organoids (FIG. 4c), in accordance with the known gradual replacement of CK14 by CK19 in differentiating ameloblasts. Moreover, the MIM-cultured organoids displayed calcium deposits (FIG. 4d; Alizarin Red S (ARS) staining), supported by transmission electron microscopy (TEM) revealing the presence of electron-dense calcium-phosphate accumulations (FIG. 4d), analogous to the formation of hydroxyapatite during amelogenesis. This mineralization outcome appeared even more prominent when the MIM-cultured organoids were incubated in an in vivo environment, i.e. following subcutaneous transplantation of 3D-printed hydroxyapatite scaffolds seeded with organoids in immunodeficient mice (FIG. 10c; ARS and Masson's Trichrome staining (TCM)).

Finally, to validate whether the ameloblast differentiation capacity is already present in the organoid-initiating epithelial cell rests of Malassez stem cells, the organoids were developed immediately in MIM and subsequently analysed their phenotype (in P1; FIG. 10d). ODAM expression was detected in the MIM-(but not TOM-) grown organoids coinciding with the almost absence of SOX2⁺ cells. AMTN and KLK4 were also detected at higher levels in MIM-versus TOM-grown organoids, while MMP20 expression was only lowly expressed by both organoid types (FIG. 10d).

### EXAMPLE 5. SINGLE-CELL TRANSCRIPTOMICS OF TOOTH ORGANOIDS ENRICHES INSIGHTS INTO AMELOGENESIS

To decipher the amelogenesis differentiation process that occurs in the tooth organoids in deeper detail, scRNA-seq analysis was performed of P4 organoids switched to MIM for 8 days (referred to as P4-switch; see FIG. 4a), and integrated the data with the scRNA-seq dataset described above (FIG. 5a).

As expected, stemness markers (e.g. SOX2, KRT15) are more prominent in the non-differentiated P4 organoid cluster, whereas ameloblast differentiation markers (e.g. AMTN, ODAM) show almost exclusive expression in the differentiated P4-switch organoids (FIG. 5b), all concordant with the findings above. Analogously, the newly identified EGR1 and ATF3 are mainly expressed in the non-differentiated P4 organoid cluster, while the tooth development marker PITX2 was found in both stemness and differentiated organoid groups (FIG. 11a).

Looking more broadly at gene expression differences using DEG analysis revealed that P4 and P4-switch organoid clusters clearly display different gene signatures, thereby exposing interesting (new) markers (FIG. 5c). Among others, ornithine decarboxylase 1 (ODC1), a gene involved in cell cycle regulation and proposed as a marker of dental epithelium (moreover aberrantly expressed in specific odontogenic tumours), is higher expressed in P4 versus P4-switch organoids (FIG. 5c). In addition to KRT15 and ATF3 belonging to the top 10 upregulated DEGs in P4 organoids (FIG. 5c), other undifferentiated epithelial cell markers are also distinctly expressed in P4 versus P4-switch organoids, including death-associated protein-like 1 (DAPL1) and tissue inhibitor of metalloproteinases 1 (TIMP1) (FIG. 5c), both described markers of mouse dental epithelium. On the other hand, in addition to AMTN and ODAM surfacing in the top 10 DEGs of P4-switch as compared to P4 organoids (FIG. 5c), laminin subunit gamma 2 (LAMC2) and laminin subunit alpha 3 (LAMA3), both expressed in mature ameloblasts in mouse incisor and essential for amelogenesis in humans (with mutations linked to amelogenesis imperfecta), are highly upregulated in P4-switch organoids (FIG. 5c), further validated by RT-qPCR (FIG. 11b). Follicular dendritic cell secreted peptide (FDCSP), reported to bind to hydroxyapatite, is also distinctly expressed in the P4-switch organoids (FIG. 5c).

GO analysis revealed enriched ‘negative regulation of cell differentiation’ in the straight P4 organoids when compared to P4-switch organoids (FIG. 11c). In the reverse comparison, GO analysis exposed enrichment of ‘epithelial cell differentiation’, ‘biomineral tissue development’, ‘odontogenesis’ and ‘calcium-mediated signalling’ in P4-switch versus P4 organoids (FIG. 5d). Interestingly, also TGFβ-associated processes are upregulated (FIG. 5d), in line with the enrichment of ‘negative regulation of SMAD protein signal transduction’ in the non-differentiated organoids (FIG. 11c), and the knowledge that the TGFβ pathway plays an important role in ameloblast differentiation [Benedete (2008) Pediatr. Dev. Pathol. 11, 206-212].

Gene set enrichment analysis (GSEA) was performed which exposed several important differentiation (amelogenesis) pathways in P4-switch versus P4 organoids. Firstly, mineralization hallmarks (tooth, enamel) are significantly enriched in P4-switch organoids. In addition, calcium-signalling pathways, highly important during amelogenesis, were found significantly associated with the P4-switch organoids such as the hallmarks ‘calmodulin binding’, ‘store-operated calcium entry’ and ‘calcium mediated signalling’. Further interestingly, GSEA revealed significant enrichment of TGFβ signalling hallmarks in P4-switch versus P4 organoids, more specifically TGFβ (receptor) signalling and TGFβ (particularly TGFβ1/3) production, in line with the importance of the TGFβ pathway in amelogenesis [Benedete (2008) Pediatr. Dev. Pathol. 11, 206-212].

Regulon analysis exposed higher activity of the signal transducer and activator of transcription 2 (STAT2) gene-regulatory network in P4-switch than P4 organoids (FIG. 5e). STAT2 is specifically found in ameloblasts (reported in neonatal rat molars) and positively targets AMTN, the ameloblast-related LAMC2 and LAMB3, as well as genes associated with TGFβ signalling (TGFβ3, TGFβR2) (FIG. 5e). Also, avian musculoaponeurotic fibrosarcoma (MAF), specifically expressed in ameloblasts (reported in mouse incisor tooth germs and representing an essential regulator of AMELX secretion during amelogenesis, shows higher regulon activity in P4-switch than P4 organoids (FIG. 5e). MAF is predicted to positively regulate fibronectin (FN1) and RUNX2, genes related to ameloblast differentiation, and TGFβ signalling-associated SMAD3, SMAD6 and TGFβ-induced (TGFβI), an activated form of the TGFβ1 ligand (FIG. 5e). RUNX2 expression has been reported in ameloblasts during the late secretory and maturation stages and its deletion suppresses enamel maturation. In addition, TGFβ1 affects enamel mineralization by modulating RUNX2. TGFβI is also an important predicted target gene of Forkhead Box C2 (FOXC2), and FOXC2 regulon activity was found higher in P4-switch than P4 organoids (FIG. 5e). FOXC2 is highly expressed during craniofacial development, but its exact role during tooth development and differentiation is unknown. FOXC2 is also predicted to positively regulate LAMC2 and MSX1, a highly conserved transcription factor well-known to regulate tooth formation, and causing tooth agenesis in humans when mutated. Finally, SOX4 and HMGA2 regulons are prominently activated in P4-switch organoids (FIG. 11d). SOX4 expression has been reported in dental epithelial stem cells and in inner enamel epithelium (at the cap stage in mouse) and targets PITX2, while HMGA2 is involved in early tooth formation and stem cell marker (e.g. SOX2) expression (hence, plausibly associated with enlarged and supernumerary teeth when truncated), and predicted targets include FN1 and LAMA3 (FIG. 11d). Pseudotime trajectory analysis (using Monocle3) projected a potential developmental path from P1-P4 to P4-switch clusters (FIG. 11e). Intriguingly, the trajectory passes through a particular subcluster of the P4-switch organoids (FIG. 11e, encircled), likely representing a transitional stage as supported by the concurrent expression of stemness/development markers (SOX2, KRT15, PITX2) and differentiation markers (AMTN, ODAM) in this subcluster (see FIG. 5b; FIG. 11a). Interestingly, regulons controlling ameloblast differentiation (PITX1, DLX3, MEIS1) are especially active in this subcluster (FIG. 11f). PITX1 is required for proper tooth formation, and has been described in secretory stage ameloblasts. DLX3 promotes the expression of EMPs during amelogenesis, and MEIS1 has been shown to bind to DLX3.

In a final analysis of the scRNA-seq data, STRING was applied to in silico predict protein-protein interactions. Using the top 40 DEGs in P4-switch versus P4 organoid clusters, it is projected that AMTN and ODAM closely interact (FIG. 11g). AMTN is also predicted to cooperate with C4orf26, an ECM acidic phosphoprotein suggested to play a key role in enamel mineralization and crystal nucleation. In addition, the STRING analysis proposed interaction of AMTN with LAMB3 suggesting a role in cell-matrix attachment, in line with previously proposed interactions of AMTN with laminins localized in the epithelial basement membrane of the ECM. In the predicted network, LAMB3 interacts with LAMA3 and LAMC2. AMTN is also proposed to network with FN1, at present not reported. Moreover, FN1 is predicted to interact with TGFβI and ITGB6. ITGB6 is known to activate TGFβ1 by binding to arginine-glycine-aspartic acid (RGD) motifs present in ECM proteins such as FN1. GO-Biological Process analysis of these particular top 40 DEGs in P4-switch organoids confirmed key features of biomineral tissue development, odontogenesis and enamel mineralization, as well as of TGFβ signalling, the latter further stressed by KEGG pathway analysis (FIG. 11h).

### EXAMPLE 6. TGFβ FORTIFIES AMELOGENESIS IN TOOTH ORGANOIDS AND TRIGGERS PERIODONTAL LIGAMENT-LIKE DIFFERENTIATION

The above analyses exposed the enrichment of TGFβ pathway processes in tooth organoids subjected to ameloblast differentiation, in line with the proposed key role of TGFβ in amelogenesis. To assess the impact of the TGFβ pathway, organoids grown in TOM (P5) were switch to MIM with or without TGFβ (FIG. 6a). Immunofluorescence analysis revealed that addition of TGFb further upregulated the expression of ODAM (FIG. 6a), supported by gene expression interrogation also showing increased expression of AMTN (FIG. 6b). The effect of TGFβ was blocked by the simultaneous addition of a TGFβ receptor inhibitor (LY2109761, further referred to as TGFβinh), thereby demonstrating the specificity of the effect observed (FIG. 6b). Intriguingly, adding TGFβinh to MIM-cultured organoids (i.e. without adding exogenous TGFβ) strongly reduced the upregulated ODAM and AMTN expression in MIM (FIG. 6b), indicating the presence and implication of endogenous TGFβ signalling in the differentiation process, in line with the findings above (FIG. 5d) and corroborated by the increase in expression of TGFβ1 and its receptors TGFβR1 and TGFβR2 in MIM culture (FIG. 12a). Together, the data demonstrate that TGFβ further advances the amelogenesis-mimicking differentiation in the tooth organoids.

During tooth root development, Hertwig's epithelial root sheath (HERS), from which epithelial cell rests of Malassez is eventually derived, undergoes EMT to develop to participate in periodontal ligament formation. It has previously been proposed that this EMT process is regulated by TGFβ, thereby triggering HERS/epithelial cell rests of Malassez cells to switch phenotype toward periodontal ligament cells. Addition of TGFβ indeed further increased the expression of VIM in the epithelial (CK5+) organoids (FIG. 6a) and also significantly stimulated the expression of the PERIODONTAL LIGAMENT-specific genes periostin (POSTN) and collagen type III alpha 1 chain (COL3A1) (FIG. 6c).

### EXAMPLE 7. THE PRESENCE OF TOOTH MESENCHYMAL CELLS TRIGGERS AMELOBLAST DIFFERENTIATION IN THE EPITHELIAL ORGANOIDS

Given the importance of mesenchyme-epithelium interactions during tooth development including ameloblast differentiation/amelogenesis, it was investigated whether addition of dental mesenchymal cells had an impact on ameloblast differentiation of the epithelial organoids. It was chosen to use DPSCs to mimic early stages of tooth development in which DPSC-derived odontoblasts are in close contact with ameloblasts. The DPSCs, isolated, grown and characterized using well-defined standard protocols, were combined with organoid-derived epithelial stem cells in a layered approach, thereby forming composite organoids (assembloids) which were cultured in a mixture of TOM and the DPSC growth medium αMEM (FIG. 6d). The hybrid epithelial-mesenchymal composition was confirmed by CK5-VIM immunofluorescence analysis (FIG. 6d), revealing VIM⁺ mesenchymal cells in the inner part and CK5⁺ epithelial cells at the outer zone of the assembloids (FIG. 6d), and by developing assembloids using eGFP-expressing DPSCs (FIG. 12b).

Whereas ODAM is not present in the straight (pure) epithelial organoids cultured in TOM (see above and FIG. 12c), it is expressed in the assembloids (FIG. 6d). This induction is not due to the addition of αMEM to TOM (FIG. 12c). Interestingly, the epithelial cells neighbouring the DPSCs express ODAM, whereas the cells at the outside border of the assembloids (thus, not in direct contact with the mesenchymal cells while more exposed to the (stem cell) medium) do not (FIG. 6d). These findings indicate that the presence of (and even more, the close interface with) mesenchymal (stem) cells drives epithelial stem cells into ameloblast differentiation. Addition of TGFβinh completely abolished ODAM protein expression in the assembloids (FIG. 6d) and reduced the expression of AMTN (FIG. 12d), indicating the involvement of endogenous TGFβ signalling in the observed effects. TGFβ pathway components are indeed expressed in the assembloid culture (FIG. 12e); the ligand(s) may originate from the epithelial cells (see FIG. 12a), further upregulated by the presence of mesenchymal cells, or may be additionally produced by the mesenchymal cells since both dental cell types have been shown to produce TGFβ [Kobayashi-Kinoshita et al. (2016) Sci. Rep. 6, 33644].

### EXAMPLE 8. MATERIAL & METHODS

### Isolation and Dissociation of Dental Follicle

Third molars, predominantly unerupted, were extracted from adolescent patients (Table 2) at the ‘Oral and Maxillo-Facial Surgery-Imaging & Pathology (OMFS-IMPATH)’ unit of University Hospitals (UZ) Leuven after informed consent. The study was approved by the Ethics Committee Research UZ/KU Leuven (13/0104U). For sample collection, the gingiva was pushed aside after which the bone was perforated and the third molars with associated dental follicles were carefully isolated (without the visually distinct gingiva). dental follicle tissue was diligently peeled from the tooth and collected in Eagle's Minimum Essential Medium (αMEM; Sigma-Aldrich) supplemented with 10% foetal bovine serum (FBS; Sigma-Aldrich), 1% penicillin-streptomycin (Gibco) and 0.5% fungizone (Amphotericin B; Gibco). Following short rinsing steps in 70% ethanol and phosphate-buffered saline (PBS; Gibco), tissue was minced into small (˜1 mm²) fragments, and further dissociated using collagenase VI (3 mg/ml; Thermo Fisher Scientific) and dispase II (4 mg/ml; Sigma-Aldrich) for 2 h at 37° C., while regularly pipetting up and down. The single cells and few small cell clusters were collected through a 40 μm cell strainer (Corning) while removing the remaining larger and fibrous tissue fragments.

### Establishment and Passaging of Tooth Organoid Culture

The dissociated dental follicle cell material was resuspended in a mixture of serum-free defined medium (SFDM; Thermo Fisher Scientific; Table 4) and growth factor-reduced Matrigel (Corning) in a 30:70 ratio, which was plated in 48-well plates at 20,000 cells per 20 μL drop. After solidification, tooth organoid medium (TOM; Table 1), unless indicated otherwise, was supplemented. ROCK inhibitor (RI; 10 μM; Merck Millipore) was added the first day of seeding (or passaging). Organoid cultures were kept at 37° C. in a 1.9% CO₂ incubator, and medium was refreshed every 2 to 3 days, each time supplemented with fungizone (0.1%).

TABLE 4
Serum-free defined medium (SFDM; pH 7.3)

| | | | | Catalogue |
|---|---|---|---|---|
| Name | Concentration | | Supplier | Number |
| Sterile H2O | | | | |
| DMEM 1:1 F12 | 16.8 | g/L | Invitrogen | 074-90715A |
| without Fe | | | | |
| Transferrin | 5 | mg/L | Serva | 36760.01 |
| Insulin from | 5 | mg/L | Sigma-Aldrich | I6634 |
| bovine pancreas | | | | |
| Penicillin | 35 | mg/L | Sigma-Aldrich | P3032 |
| Streptomycin | 50 | mg/L | Sigma-Aldrich | S6501 |
| Ethanol | 600 | μL/L | Fisher Chemical | E/0650DF/15 |
| absolute, ≥99.8% | | | | |
| (EtOH) | | | | |
| Catalase from | 50 | μL/L | Sigma-Aldrich | C100 |
| bovine liver | | | | |
| NaHCO3 | 1 | g/L | Merck | 106329 |
| Bovine serum | 5 | g/L | Serva | 47330.03 |

albumin (BSA)

The organoid cultures were passaged every 10 to 14 days. Matrigel droplets were collected using ice-cold SFDM, and organoids dissociated using TrypLE (containing 5 μM RI; Thermo Fisher Scientific) and mechanical trituration. Remaining large organoid fragments were allowed to sediment and the supernatant, containing single cells and small fragments, seeded as described above. A split ratio of 1:6 was applied once the culture reached stable growth (typically from P2-P4). Organoids were cryopreserved and stored in liquid nitrogen.

To assess clonal derivation, dissociated single organoid cells were transduced with the lentiviral vector LV-eGFP during 30 min at 37° C., resulting in 60% eGFP⁺ cells as analysed by flow cytometry. The resulting mixture of eGFP⁺ and eGFP⁻ cells was seeded in organoid culture as described above, and cultures analysed 14 days later using brightfield and epifluorescence microscopy (Axiovert 40 CFL; Zeiss).

FACS isolation of ITGα6^{+/-} cells from dental follicle

Primary dental follicles were dissociated into single cells as described above. Cells were incubated with PE-anti-ITGα6 antibody (1:5; Cat.no 555736; BD Biosciences) and rinsed, both performed in TOM supplemented with fungizone (0.1%) and RI (10 μM). ITGα6⁺ and ITGα6⁻ cells within the living (DAPI-negative) population were sorted in TOM (supplemented with fungizone and RI) using a BD Influx (BD Biosciences), and seeded at 7,500 cells per 20 μL Matrigel droplet as mentioned above. RI (10 UM) was added to the cultures for 1 week.

### In Vitro Differentiation of the Dental Follicle-Derived Epithelial Organoids

Organoids (or dissociated dental follicle) were cultured in mineralization-inducing medium (MIM; Table 3; time schedule, see FIG. 4a and FIG. 10d) as described above. Recombinant human TGFβ1 (10 ng/ml; R&D) and the selective TGFβ receptor 1/2 inhibitor LY2109761 (5 μM; Selleckchem) were added when indicated.

### In Vivo Transplantation of the Dental Follicle-Derived Epithelial Organoids

Matrigel (10 μl) with dissociated organoid cells (150,000) was pipetted into custom-made 3D-printed hydroxyapatite constructs (Sirris) which were subcutaneously transplanted in immunodeficient nu/nu mice (Janvier Labs), as in detail described in [Bronckaers et al. (2021) Methods Mol. Biol. 2206, 223-232.]. After 4 weeks, implants were resected and subjected to 48h-fixation in 4% paraformaldehyde (PFA) (Sigma-Aldrich), paraffin-embedding, 24h-decalcification, 7-μm sectioning and Alizarin Red S (ARS) or Masson's Trichrome (TCM) staining as described in Bronckaers et al. (2021) Methods in Mol. Biol. 2206, 223-232. The study was approved by the Ethical Committee on Animal Experiments (ECAE) of Hasselt University (protocol 202044).

### Dental Pulp Stem Cell Culture

DPSCs were obtained as in detail described and characterized in About et al. (2000) Am. J. Pathol. 157, 287-295. In short, dental pulp was collected from the extracted wisdom teeth (after careful removal of the apical papilla), minced and fragments cultured in T25 flasks (Corning) in αMEM supplemented with 10% foetal bovine serum (FBS) and 1% L-glutamine (Gibco). When 70-80% confluence was reached, cells were trypsinized and re-plated at 150,000 cells per T75 flask, and used at early passage (˜P5) for assembloid creation. For GFP labelling, DPSCs were transduced with the lentiviral vector LV-eGFP as described above.

### Development and Culture of Assembloids

Organoid and DPSC cultures were dissociated into single cells, and mixed in a round-bottom low-attachment plate (96-well; Greiner) using a layered approach [Nakao et al. (2007) Nat. Methods 4, 227-230]. First, DPSCs (5×10⁴ cells) were sedimented by centrifugation (300 g for 1 min at 4° C.), followed by deposition of the organoid-derived cells (1×10⁵; at 300 g and 4° C. for 2 min). The cells were layered in 10% Matrigel and 90% of a 1:1 mixture of TOM (i.e. organoid growth medium) and αMEM (i.e. DPSC growth medium), and then incubated for 24 h at 37° C. in 5% CO₂. The formed aggregate was re-plated into a 48-well plate in a 20 μL Matrigel (70%) droplet as described above to generate the assembloid, further cultured in TOM+αMEM with or without the TGFβ receptor inhibitor LY2109761 (5 μM) as indicated.

### Histochemical and Immunostaining Analysis

Primary dental follicle tissue, organoids and assembloid were fixed in 4% PFA for 1h, embedded in paraffin, and sections subjected to hematoxylin and eosin (H&E), immunofluorescence or ARS staining. Antigen retrieval (10 mM citrate, pH6) and permeabilization (0.1% Triton X-100; Sigma-Aldrich) were performed. After incubation with primary and secondary antibodies (Table 5), sections were mounted with Vectashield (DAPI; Vector Laboratories) or DPX mountant (Sigma-Aldrich). Analysis was done using a Leica DM5500 epifluorescence microscope or a Zeiss Axioimager epifluorescence microscope. ImageJ software was used to quantify immunoreactive signal intensity and the ‘corrected total organoid fluorescence’ (CTOF) (=integrated density−(area of organoid×mean fluorescence of background readings).

TABLE 5
Antibodies used for immunohistochemical/immunofluorescence
staining
Primary antibodies

| | | | Clone or Catalogue | |
|---|---|---|---|---|
| Antigen | Host | Supplier | Number | Dilution |
| AMELX | mouse | Santa Cruz | sc-365284 | 1:100 |
| CC3 | rabbit | Sigma | AB3623 | 1:100 |
| CD44 | mouse | Abcam | ab34485 | 1:200 |
| CD90 | rabbit | Abcam | ab133350 | 1:100 |
| CK5 | rabbit | Bioledgend | Poly19055 | 1:100 |
| CK14 | mouse | Thermo Fisher | LL002 | 1:200 |
| | | Scientific | | |
| CK19 | mouse | Dako | M772 | 1:50 |
| KI67 | mouse | BD Bioscience | 556003 | 1:100 |
| ODAM | rabbit | Proteintech | 16509-1-AP | 1:200 |
| P63 | rabbit | Abcam | ab124762 | 1:1000 |
| SOX2 | rabbit | Abcam | ab92494 | 1:2000 |
| VIM | mouse | Dako | V9 | ready-to-use |

Secondary antibodies

| | | | Catalogue | |
|---|---|---|---|---|
| Antigen | Host | Supplier | Number | Dilution |
| mouse IgG (Alexa | donkey | Thermo Fisher | A-21202 | 1:1000 |
| 488) | | Scientific | | |
| mouse IgG (Alexa | donkey | Thermo Fisher | A-31570 | 1:1000 |
| 555) | | Scientific | | |
| rabbit IgG (Alexa | donkey | Thermo Fisher | A21206 | 1:1000 |
| 488) | | Scientific | | |
| rabbit IgG (Alexa | donkey | Thermo Fisher | A-31572 | 1:1000 |
| 555) | | Scientific | | |

### Transmission Electron Microscopy

Organoid samples were prepared for transmission electron microscopy (TEM). In short, samples were fixed in glutaraldehyde/osmium tetroxide, dehydrated, embedded in epoxy resin, and cut into 40-70 nm sections. TEM analysis was performed with the JEM1400 transmission electron microscope (JEOL) equipped with an Olympus SIS Quesmesa 11 Mpxl camera, or the Philips EM208 S electron microscope (Philips) equipped with the Morada Soft Imaging System camera with corresponding iTEM-FEI software (Olympus SIS).

### Gene Expression Analysis by RT-qPCR

RNA was extracted from dissociated dental follicle, organoids and assembloids using the GenElute Mammalian Total RNA Miniprep Kit (Sigma-Aldrich) according to the manufacturer's instructions. RNA was reverse-transcribed (RT) using the Superscript III First-Strand Synthesis Supermix (Thermo Fisher Scientific) and the resultant cDNA samples were analysed with SYBR Green-based quantitative PCR (qPCR) using the StepOnePlus Real-Time PCR System (AB Applied Biosystems). Forward and reverse primers (Table 6) were designed using PrimerBank and PrimerBlast. Glyceraldehyde-3-phosphate dehydrogenase (GAPDH) was included as housekeeping gene. Relative gene expression levels were calculated as ΔCt (Ct target−Ct housekeeping gene) and compared to control (see figure legends).

### Single-Cell RNA-Sequencing Analysis

Primary dental follicle tissue from two individual patients (see Table 2), and derived organoids at P1 and P4, or switched to MIM (P4-switch), were dissociated into single cells (as described above) and subjected to scRNA-seq analyses using 10× Genomics, according to manufacturer instructions. Libraries were generated using the Chromium Single Cell 3′ v2 Chemistry Library Kit, Gel Bead & Multiplex Kit (10× Genomics), and sequenced on NovaSeq6000. After quality control, raw sequencing reads were demultiplexed, aligned to the human reference genome GRCh38 and processed to a matrix representing the UMI's per cell barcode per gene using CellRanger (v3; 10× Genomics). Downstream analysis was performed in R (v.3.6.1) using Seurat (v.3.0).

First, data from the primary dental follicle tissue, P1 and P4 organoids were integrated and analysed, and subsequently data from P4-switch organoids were added for a next analysis (further referred to as Integration 1 and Integration 2, respectively). Dead cells and potential doublets (i.e. with <300 genes or >10,000 genes, >150,000 unique molecular identifiers (UMI) and >15% mitochondrial RNA) were removed (FIG. 8b), resulting in a total of 22,317 cells in Integration 1 and 27,851 cells in Integration 2. Integration anchors were identified using the FindIntegrationAnchors function with default parameters and dims=1:30, and data were integrated across all features. Next, expression levels were scaled, centered and subjected to principal component analysis (PCA). The top 30 PCs were selected and used for UMAP dimensionality reduction. Clusters were calculated by the FindClusters function with a resolution set to 2 and 0.1 for Integration 1 and Integration 2, respectively. Differential gene expression was calculated for each cluster using the MAST package (v.1.12.0). All clusters were annotated based on reported dental follicle and ERM markers and on recent scRNA-seq studies of mouse teeth. Marker genes were defined using FindAllMarkers in Seurat.

Gene ontology analysis (GO) of biological processes was done in Panther using significant differentially expressed genes (DEGs; FDR≤0.05 and log FC≥0.25). Gene-regulatory networks (regulons) were identified using SCENIC (pySCENIC; v.0.9.15) in Python (v.3.6.9). In short, co-expression modules were generated and regulons inferred (with parameters and hg38__refseq-default r80_10 kb_up_and_down_tss.mc9nr.feather and hg38__refseq-r80_500 bp_up_and_100 bp_down_tss.mc9nr.feather motif collections) resulting in a matrix of AUCell values that represent the activity of each regulon in each cell. The AUCell matrix was imported into Seurat and regulons were projected on the integrated UMAP plot.

Gene-set enrichment analysis (GSEA; v.4.1.0) was performed on P4 and P4-switch organoids using normalized expression data. Gene sets (hallmarks) tested were obtained from the Molecular Signatures Database (MSigDB; v.7.2).

To predict protein-protein interactions with STRING (v.11.0), the top 40 DEGs of P4-switch organoids versus P4 organoids were used. The cluster analysis was subdivided in three colours by kmeans. The minimum required interaction score was set as medium confidence (0.4).

Finally, the pseudotime trajectory was projected onto the integrated UMAP dimensional reduction generated previously with Seurat (P1, P4 and P4-switch organoids) using the Monocle3 (v1.0.0) package's learn_graph and plot_cells functions.

Raw sequencing data are available at ArrayExpress (accession number E-MTAB-10596).

### STATISTICAL ANALYSIS

Statistical analysis was performed using GraphPad Prism (v.9.0.0). (Un-) paired two-tailed t-student test was applied for comparison of 2 groups or two-way analysis of variance (ANOVA) for multiple comparisons followed by Sidak's test for Multiple Comparison. Statistical significance was defined as P≤0.05.

1. A method for developing and growing tooth organoids comprising the steps of:
dissociating tooth tissue comprising dental epithelial stem cells;
seeding the dissociated cells in a scaffold mimicking an extracellular matrix; and
growing and passaging the cells in a medium suitable for organoid growth,), thereby obtaining and expanding organoids, characterised in that the medium comprises:
a WNT agonist,
a key nicotinamide adenine dinucleotide (NAD+) intermediate,
an activin receptor-like kinase (ALK) inhibitor,
a p38 MAP kinase inhibitor,
a bone morphogenetic protein (BMP) inhibitor,
a fibroblast growth factor (FGF),
an insulin-like growth factor,
a free-radical scavenger,
a hedgehog signalling agonist,
an adenylate cyclase-CAMP agonist,
B27,
L-glutamine, and
N2.

2. The method according to claim 1 wherein the medium is a serum free medium.

3. The medium according to claim 1 or 2, wherein the medium does not comprise epidermal growth factor (EGF).

4. The method according to any one of claims 1 1 to 3, wherein the medium comprises:
between 175 and 225 ng/ml, or 200 ng/ml R-spondin 1 (RSPO1) and between 175 and 225 ng/ml, or 200 ng/ml WNT3A,
between 9 and 11 mM or 10 mM nicotinamide,
between 0.45 and 0.55 UM or 0.5 μM A83-01,
between 9 and 10 UM or 10 μM SB202190,
between 90 and 110 ng/ml or 100 ng/ml Noggin,
between 175 and 225 ng/ml, or 200 ng/ml FGF8,
between 90 and 110 ng/ml or 100 ng/ml FGF10,
between 17.5 and 22.5 ng/ml, or 20 ng/ml FGF2,
between 90 and 110 ng/ml or 100 ng/ml IGF-1,
between 1.125 and 1.375 mM or 1.25 mM N-acetyl-cysteine (NAC),
between 90 and 110 ng/ml or 100 ng/ml SHH,
between 90 and 110 ng/ml, or 100 ng/ml cholera toxin,
between 1.9% and 2.1%, or 2% B27,
between 1.75 and 2.25 mM or 2 mM L-glutamine, and
between 0.9 and 1.1%, or 1% N2.

5. The method according to any one of claims 1 to 4, wherein the stem cells, typically tooth epithelial stem cells, are isolated from dental follicle or from dental periodontal ligament.

6. The method according to any one of claims 1, 2, 4 or 5, the medium further comprising EGF, thereby inducing mesenchymal properties.

7. The method according to any one of claims 1 to 6, wherein the organoids are further cultured in a medium comprising transforming growth factor beta (TGFβ), thereby enhancing amelogenesis and periodontal ligament differentiation.

8. A tooth organoid comprising epithelial cells from tooth tissue and expressing amelogenin (AMELX), obtainable by the method according to any one of claims 1 to 5.

9. The organoid according to claim 8, which does not express ODAM and/or which does not express one or more of CD90, fibroblast activation protein alpha (FAP) and Collagen Type I alpha I (COL1A1)

10. The organoid according to claim 8 or 9, expressing cytokeratin 14 (CK14), 5 (CK5) and/or expressing one or more of CD44, TP63, SOX2, ITGA6, BMP4 and KRT15.

11. A differentiated tooth organoid obtainable by the method according to claim 6 or 7 comprising epithelial cells from tooth tissue and producing electron-dense calcium-phosphate accumulations, expressing ODAM and AMELX.

12. The differentiated tooth organoid according to claim 11, staining positive for Alizarin Red Staining and/or expressing AMTN, KLK4 and CK19, and/or expressing one or more of LAMC2, LAMA3, LAMB3, FDCSP, STIM1, CALB2 and TGFβI.

13. A hybrid organoid obtainable by the method according to any one of claims 1 to 7, comprising epithelial cells from tooth tissue and mesenchymal cells from dental tissue producing (pulp) electron-dense calcium-phosphate accumulations, expressing ODAM and AMTN.

14. A medium for the development, growth and culture of epithelial organoids from human tooth tissue, wherein the medium comprises:
a WNT agonist,
a key nicotinamide adenine dinucleotide (NAD+) intermediate,
an activin receptor-like kinase (ALK) inhibitor,
a p38 MAP kinase inhibitor,
a bone morphogenetic protein (BMP) inhibitor,
a fibroblast growth factor (FGF),
an insulin-like growth factor,
a free-radical scavenger,
a hedgehog signalling agonist,
an adenylate cyclase-CAMP agonist,
B27,
L-glutamine and
N2.

15. The medium according to claim 14, which is a serum free medium.

16. The medium according claim 14 or 15 wherein the medium does not comprise epidermal growth factor (EGF), and wherein.

17. The medium according to any one of claims 14 to 16 comprising:
between 175 and 225 ng/ml, or 200 ng/ml R-spondin 1 (RSPO1) or comprising between 150 and 250 ng/ml, or between 175 and 225 ng/ml, or 200 ng/ml WNT3A,
between 9 and 11 mM or 10 mM nicotinamide,
between 0.45 and 0.55 UM or 0.5 μM A83-01,
between 9 and 10 UM or 10 UM SB202190,
between 90 and 110 ng/ml or 100 ng/ml Noggin,
between 75 and 225 ng/ml, or 200 ng/ml FGF8,
between 90 and 110 ng/ml or 100 ng/ml FGF10,
between 17.5 and 22.5 ng/ml, or 20 ng/ml FGF2,
between 90 and 110 ng/ml or 100 ng/ml IGF-1,
between 1.15 and 1.35 mM or 1.25 mM N-acetyl-cysteine (NAC),
between 90 and 110 ng/ml or 100 ng/ml SHH,
between 90 and 110 ng/ml or 100 ng/ml cholera toxin,
between 1.9% and 2.1%, or 2% B27,
between 1.75 and 2.25 mM or 2 mM L-glutamine, and
between 0.9 and 1.1% or 1% N2.

18. Use of a medium according to any one of claims 14 to 17, for the development, growth and culture of epithelial organoids from human tooth tissue.
